(19) United States
(12) Patent Application Publication
(10) Pub. No.: US 2022/0195424 A1
(43) Pub. Date: Jun. 23, 2022

(54) Chemical Capping for Template Switching

(71) Applicant: New England Biolabs, Inc., Ipswich, MA (US)

(72) Inventors: Ivan R. Correa, Jr., Hamilton, MA (US); Shengxi Guan, Stoneham, MA (US); Madalee G. Wulf, Ipswich, MA (US); Nan Dai, Ipswich, MA (US); Sean Maguire, Gloucester, MA (US)

(73) Assignee: New England Biolabs, Inc., Ipswich, MA (US)

(21) Appl. No.: 17/610,081

(22) PCT Filed: May 6, 2020
(86) PCT No.: PCT/US2020/031653

§ 371 Date: Nov. 9, 2021

Related U.S. Application Data
(60) Provisional application No. 62/846,207, filed on May 10, 2019.

(51) Int. Cl. C12N 15/10; C12Q 1/6806

(52) U.S. Cl. CPC C12N 15/1096; C12Q 1/6806

(57) ABSTRACT

Provided herein is a method for chemically capping polynucleotides having a 5′ monophosphate. In some embodiments the method may comprise: combining an activated nucleoside 5′ mono- or poly-phosphate with a population of polynucleotides that comprises polynucleotides having a 5′ monophosphate, to produce a reaction mix; and incubating the reaction mix to produce reaction products that comprise a polynucleotide and a 5′ nucleoside cap, linked by a 5′ to 5′ polyphosphate linkage. The chemical capping method described herein can be incorporated into a variety of cDNA synthesis methods.

### BACKGROUND

Template-switching (TS) events are generated in a homology-dependent manner Specifically, the growing complementary DNA (cDNA) strand synthesized by a reverse transcriptase (RT) dissociates from its original template and re-associates with another template at the 3′ end of the cDNA.

TS has been used to create a cDNA that contains a sequence complementary to an oligonucleotide of known sequence also known as the template-switching oligonucleotide (TSO) and a target RNA as a step in generating a sequencing library. In vitro methods of TS have utilized the naturally occurring m⁷Gppp cap at the 5′ end of eukaryotic messenger RNA (mRNA) to make cDNA libraries of mRNA. Most RNAs including prokaryotic mRNAs are naturally uncapped. However, Vaccinia Capping Enzyme (New England Biolabs, Ipswich, Mass.) can cap those RNAs that have a diphosphate or triphosphate to form cDNA libraries from these RNAs. Unfortunately, many important regulatory RNAs, such as microRNAs, and fragments of mRNA that have lost the terminal capped nucleotides, do not have a diphosphate or triphosphate at the 5′ end and therefore cannot be enzymatically capped.

Another problem associated with TS is that of bias. The RTs that are used for TS appear to discriminate between RNAs with different terminal nucleotides at the 5′ end of the RNA resulting in bias in the representation of input RNA abundance based on sequence read frequencies.

It would be desirable to be able to use TS for any RNA in a total RNA population. This would require accessing those RNAs with a single phosphate or no phosphate at the 5′ end and doing so with the minimum of bias associated, regardless of the identity of the 5′ terminal nucleotide on the RNA.

It would also be desirable to be able to use TS for DNAs, in particular for the analysis of degraded and/or fragmented DNA having a high single-stranded content, such as ancient DNA, environmental DNA, forensic DNA, circulating DNA (e.g., exosomes), denatured DNA and viral DNA. Several of these DNAs may be used as biomarkers and in medical diagnosis applications.

### SUMMARY

Provided herein is a method for chemically capping a population of polynucleotides having a 5′ monophosphate. The method may include: (a) combining an activated nucleoside 5′ mono- or poly-phosphate with a population of polynucleotides that comprises polynucleotides having a 5′ monophosphate, to produce a reaction mix; and (b) incubating the reaction mix to produce reaction products that each comprise a polynucleotide linked to a 5′ nucleoside cap by a 5′ to 5′ polyphosphate linkage.

The population of polynucleotides may be RNAs. For example, the RNA population may include one or more of RNA species selected from small RNAs, microRNAs (miRNAs), transfer RNAs (tRNAs), long noncoding RNAs (lncRNAs), and fragmented mRNAs. The polynucleotides in the population may be single-stranded or partially single-stranded DNAs (ssDNAs). The population of polynucleotide may be a population of polynucleotides of a liquid biopsy, a population of polynucleotides of a cell, a population of polynucleotides of a virus, or a population of polynucleotides of formalin-fixed, paraffin-embedded tissue or a population of polynucleotides of another source described herein.

The polynucleotides described above having a 5′ monophosphate may be the product of enzymatic addition of a single phosphate to the 5′ end of a polynucleotides having no terminal phosphate. This may be achieved using a polynucleotide kinase.

The polynucleotides having a 5′ monophosphate may be the products of a decapping reaction of 5′ capped RNAs. In these embodiments the decapping may done using an enzyme selected from a deadenylase, an apyrase, a 5′RNA polyphosphatase (RppH), an Nudix phosphohydrolase, a tobacco acid polyphosphatase, a member of the histidine triad (HIT) superfamily of pyrophosphatases, a DcpS, a Dcp1-Dcp2 complex, a NudC, or an aprataxin (APTX).

In any embodiment the reaction mix can have a pH of in the range of pH 5-pH 6.5.

With respect to the capping reaction, the activated nucleoside 5′ mono- or poly-phosphate may include an imidazole moiety where a nucleophilic substitution reaction displaces the imidazole. For example, in some embodiments, the activated nucleoside 5′ mono- or poly-phosphate may be a phosphoroimidazole-NMP, -NDP or -NTP and the method may comprise incubating the reaction mix for less than 10 hours at a temperature of 30° C.-60° C. at an acidic pH for example at 50° C. for 5 hours, 37° C. for 4 hours, or room temperature for 4 hours at a pH of in the range of pH 5-pH 6.5 to displace the imidazole and form the 5′ capped polynucleotides.

The 5′ nucleoside cap may be of formula (I):

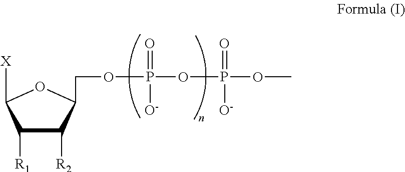

wherein, X is a nitrogenous base; R1 and/or R2═O-alkyl, halogen, a linker, hydrogen or a hydroxyl; n is any integer from 1-9; and the polynucleotide cap is a single stereoisomer or plurality of stereoisomers of one or more of the compounds described by Formula (I) or a salt or salts thereof.

In any embodiment, the nitrogenous base of the 5′ nucleoside cap may be selected from the group consisting guanine, adenine, cytosine, uracil and hypoxanthine and analogs of guanine, adenine, cytosine, uracil and hypoxanthine or modifications thereof. For example, a modified nitrogenous base of the 5′ nucleoside cap may comprise a modified base selected from N6-methyladenine, N1-methyladenine, N6-2′-O-dimethyladenosine, pseudouridine, N1-methylpseudouridine, 5-iodouridine, 4-thiouridine, 2-thiouridine, 5-methyluridine, pseudoisocytosine, 5-methoxycytosine, 2-thiocytosine, 5-hydroxycytosine, N4-methylcytosine, 5-hydroxymethylcytosine, hypoxanthine, N1-methylguanine, 06-methylguanine, 1-methyl-guanosine, N2-methyl-guanosine, N7-methyl-guanosine, N2,N2-dimethyl-guanosine, 2-methyl-2′-O-methyl-guanosine, N2,N2-dimethyl-2′-O-methyl-guanosine, 1-methyl-2′-O-methyl-guanosine, N2,N7-dimethyl-2′-O-methyl-guanosine, or isoguanine. For example, in these embodiments, the nitrogenous base of the 5′ nucleoside cap may be attached to a sugar selected from a ribose or a modified ribose selected from 2′- or 3′-O-alkylribose, alkoxyribose, O-alkoxyalkylribose, fluororibose, azidoribose, allylribose, deoxyribose; an arabinose or a modified arabinose; a thioribose; an 1,5 anhydrohexitol; or a threofuranose. Accordingly, the one or more phosphates of the 5′ nucleoside cap may consist of a phosphorothioate; a phosphorodithioate; an alkyphosphonate; an arylphosphonate; a N-phosphoramidate; a boranophosphate; or a phosphonoacetate. In examples, the 5′ nucleoside cap includes guanosine.

The method described herein may further comprise reverse transcribing the reaction products where the polynucleotides include a 3′ adapter and RT priming site to initiate reverse transcription and forming cDNA. The cDNA may include a sequence at the 3′ end that is complementary to a TSO. The 3′ adaptor may include (i) a polyA tail of an RNA having a complementary polyT for priming cDNA synthesis or (ii) a ligated 3′ adaptor containing the RT priming sequence. The 3′ adapter and polynucleotide to which it is attached may be reverse transcribed in the presence of a TSO to produce cDNA that includes the complement of the TSO at the 3′ end.

The method may further include the step of amplifying the cDNA to produce an amplification product. The method may further include sequencing the cDNA or amplification product thereof.

In one example, the 5′ nucleoside cap is a guanosine triphosphate where the cDNA products corresponding to a population of polynucleotides are representative of the original population of polynucleotides without substantially bias in favor of one or more of the following: (i) those polynucleotides that have a specific nucleotide of the four nucleotides A, G, C, U or T in the first or second position at the 5′ end over any other nucleotide; or (ii) a cap comprising guanosine and one, two, three or four phosphates between the guanosine cap and the first nucleotide at the 5′ end.

Where the 5′ nucleoside cap is guanosine triphosphate then the efficiency of template switching is enhanced by at least 2-fold compared with 5′ capped polynucleotides that do not comprise an unmethylated guanosine.

A method for synthesizing a DNA complementary to a single strand target nucleic acid is also provided. The method may comprise: (a) combining an activated nucleoside 5′ mono- or poly-phosphate with a population of polynucleotides having a 5′ monophosphate, to produce a reaction mix; (b) incubating the reaction mix to produce reaction products that each comprise a polynucleotide and a 5′ nucleoside cap, linked by a 5′ to 5′ polyphosphate linkage; and (c) reverse transcribing the products of step (b) in the presence of template switching oligonucleotide (TSO) to produce cDNA that comprises the complement of the TSO at the 3′ end.

The method may further comprise: (i) ligating a 3′ adaptor to the DNA prior to step (a) or (ii) ligating a 3′ adaptor to the reaction products of (b), the adapter optionally contains a cDNA priming site, and wherein the reverse transcribing is performed using a cDNA synthesis primer that hybridizes to the adapter. In any of these embodiments, the 5′ nucleoside cap may be a guanosine triphosphate. The method may further include sequencing the cDNA.

The resulting cDNA may be representative of the starting population of DNA and approximately equivalent independent of whether the first 5′ nucleotide is A, T, G or C. The yield of cDNA comprising sequences complementary to the population of DNA and having 5′ and 3′ adapter sequences may be increased at least 2-fold for those molecules capped with 5′ guanosine triphosphate compared with those capped DNAs that do not have a 5′ cap guanosine triphosphate.

The yield of cDNA having 5′ and 3′ adapter sequences that is the product of reverse transcription of the population of DNA having a 5′ cap guanosine triphosphate may be increased at least 2-fold compared with the yield of cDNA product from a population of DNA that are not capped with a 5′ cap guanosine triphosphate.

Also provided herein is a kit. A kit may include a nucleoside 5′-phosphoroimidazolide, a capping buffer pH 5-pH 6.5, an RT and optionally a TSO in one or more different storage containers. The kit may include a plurality of modules, each module being in one or more containers, wherein a first module is a capping module comprising reagents for chemically capping a population of polynucleotide, and a second module comprising a cDNA synthesis and amplification module, wherein the second module optionally includes (i) a TSO and/or a 3′ splint adaptor.

Also provided is a composition comprising: an activated nucleoside 5′ mono- or poly-phosphate in a buffer having an acidic pH.

### DESCRIPTION OF FIGURES

The skilled artisan will understand that the drawings described below, are for illustration purposes only. The drawings are not intended to limit the scope of the present teaching in any way.

FIG. 1A-1B shows examples of activation chemistries for adding a desired nucleoside 5′ phosphate to the 5′ phosphate terminus of an RNA or DNA.

FIG. 1A shows seven examples of activated nucleoside 5′-phosphates. In these examples, the nucleoside 5′-phosphates are activated by phosphorodichloridate, phosphoramidate, phosphodiester, phophotriester P(III)-P(V) mixed anhydride, phosphite triester and 5′-H-phosphonate.

FIG. 1B shows four examples of nucleoside phosphate analogues. Analogs include ribonucleosides, deoxyribonucleosides, carbocyclic analogs and acyclic analogs.

FIG. 2A-2F schematically illustrate capping reactions in which activated nucleoside 5′-phosphates (e.g., phosphoroimidazolide precursors) are used to produce products that have a 5′ nucleoside cap, linked by a 5′ to 5′ polyphosphate linkage.

FIG. 2A shows a reaction between a nucleoside monophosphate imidazolide and a polynucleotide having a single 5′ phosphate to form a nucleoside diphosphate polynucleotide.

FIG. 2B shows a reaction between a nucleoside diphosphate imidazolide and a polynucleotide having a single 5′ phosphate to form a nucleoside triphosphate polynucleotide FIG. 2C shows a reaction between a nucleoside triphosphate imidazolide and a polynucleotide having a single 5′ phosphate to form a nucleoside tetraphosphate polynucleotide.

FIG. 2D shows a reaction between a nucleoside monophosphate imidazolide and a polynucleotide having a 5′ diphosphate to form a nucleoside triphosphate polynucleotide.

FIG. 2E shows a reaction between a nucleoside monophosphate imidazolide and a polynucleotide having a 5′ triphosphate to form a nucleoside tetraphosphate polynucleotide

FIG. 2F shows examples of imidazolide nucleoside 5′-phosphates that can be used for chemical capping. The imidazolide activation enables the attachment of virtually any nucleoside, including those with base and ribose modifications, to a 5′-phosphorylated nucleic acid. Examples are shown for guanosine, adenosine, cytidine, thymidine and inosine nucleotides. This strategy also permits the formation of capped structures with a polyphosphate bridge that has a variable length, as well the introduction of phosphate modifications (such as phosphothioesters).

FIG. 3A-3E shows the optimization of the conditions for chemical capping of a synthetic 25mer RNA.

FIG. 3A shows how a reaction between phosphoroimidazolide activated guanosine 5′-diphosphate and an RNA oligonucleotide that has a 5′-monophosphate to form products comprising a 5′ to 5′-triphosphate linkage.

FIG. 3B shows that a capping reaction performed at pH 6 was optimal. A pH 6 buffer led to a yield increase from 31% to 63% relative to pH 7 buffer.

FIG. 3C shows the capping reaction performed at different concentration of guanosine 5′-phosphoroimidazolide imGDP. Increasing the molar excess of imGDP from 10× to 1000× (relative to the RNA oligonucleotide 5′-monophosphate) led to a yield increased from 12% to 73%.

FIG. 3D shows the progress of the capping reaction at 37° C. over time. The capping reaction was performed in in pH 6 buffer, and 1000×imGDP. The results show about 85% capping yield in 4 hours or 91% in 6 hours at 37° C.

FIG. 3E shows the progress of the capping reaction at 50° C. over time. The capping reaction was performed in pH 6 buffer, and 1000×imGDP. The results show about 62% capping yield in 1 hour or 68% in 2 hours at 50° C.

FIG. 4A-4D shows four workflows for the synthesis of a cDNA first strand from a target RNA or DNA in a polynucleotide population such as occur in a cell lysate. In each workflow a different strategy was used to incorporate an oligonucleotide adaptor to the 5′ end of the cDNA. In all four workflows, the addition of an oligonucleotide adaptor at the 3′ end of the cDNA is the same and involve a step of chemical capping the 5′ end of the target RNA to enable TS by the RT. The product of TS may be followed by amplification and then sequencing.

FIG. 4A shows a workflow using a primer (“DNA Primer”) that has a portion that is either a randomized sequence or matches a segment of the 3′ end of the target RNA and another portion that has a defined sequence (primer) using a method described by Zhu, et al. (2001) Biotechniques 30: 892-7.

FIG. 4B shows a workflow that includes adding a polyA tail using a poly(A) polymerase to the 5′ chemically capped RNA, and synthesizing the cDNA using a DNA primer containing a poly(dT) segment to initiate the reverse transcription reaction using a method described by Zhu, et al. (2001) Biotechniques 30: 892-7.

FIG. 4C shows a workflow in which a 3′-adaptor is ligated to the 3′ end of the target RNA for the synthesis of a cDNA first strand that involves adding a DNA primer that hybridizes to the ligated sequence using a method described by Fuchs, et al. (2015) PLoS ONE 10: e0126049.

FIG. 4D shows a workflow in which a 3′-splint adaptor (i.e., is a double-stranded nucleic acid with a 3′-extension segment containing randomized nucleotides) can be ligated to the 3′ end of the target RNA).

FIG. 5A-5B shows that the greatest improvements in efficiency/yield and reduction in bias of TS is observed when an unmethylated guanosine cap (i.e., a cap that comprises guanosine, not 7-methyl guanosine) is present on the 5′ end of the template RNA.

FIG. 5A is a compilation of data obtained from capillary electrophoresis for each 5′ end of an RNA template specified (HO—N, p-N, m⁷GpppN and GpppN) where N represents the 5′ terminal nucleotide (A, C, G, or U) in the original 25mer RNA template sequence. Guanosine 5′ capped RNAs (GpppN) shows greater TS efficiency than 7-methylguanosine 5′ capped RNAs (m⁷GpppN) or any uncapped RNAs (HO—N or p-N) under conditions described in Example 6. For comparison purposes, the TS efficiency of GpppN was arbitrarily set to 100%. Overall, capped RNAs (m⁷GpppN and GpppN) are template switched 2- to 5-fold more efficiently than uncapped RNAs (5′-HO—N and 5′-p-N).

FIG. 5B shows the normalized distribution of TS efficiencies for each of the RNA templates used in the compilation of FIG. 5A. The normalized TS efficiency (%) for unmethylated guanosine 5′-capped RNAs (GpppN) are approximately equivalent regardless of whether first nucleotide in the target RNA is A, C, G or U, thus showing less bias than observed for other 5′ termini on the RNA template (HO—N, p-N, or m⁷GpppN).

FIG. 6 shows that the relative TS efficiencies within a given RNA template were found to be similar across various MMLV-type RTs. The 25mer RNA template sequence is represented by the nucleotide A, C, G, or U at the first position after the cap.

FIG. 7 shows that an excess of dCTP and/or the depletion of dATP in the RT reaction enhances the TS efficiency for all RNA templates regardless of the identity of the starting nucleotide (N=A, C, G, or U) of the 25mer RNA when compared with equimolar concentrations of all nucleotides. A 10-fold excess dCTP (10×dCTP) increased TS efficiency by 1.5- to 5-fold as determined by capillary electrophoresis. Data were performed in triplicate and represent mean±SD of n=2 independent experiments.

FIG. 8A-8D confirms that using sequencing reads, RNA templates chemically capped with an unmethylated guanosine (Gppp) have the most uniform (unbiased) TS regardless of the 5′ nucleotides at position 1 and 2 of the 25mer RNA template and using the same TSO (rGrGrG 3′).

FIG. 8A shows that all sequencing reads originating from a cDNA library made from guanosine 5′capped RNAs are within 2-fold of the expected value. The Sequence ID (x-axis) represents the first two variable nucleotides at the 5′ end of the RNA template sequence.

FIG. 8B shows that direct TS reverse transcription of the uncapped pool comprising of 5′-monophosphate RNAs (5′-p-NN) gave rise to a strong bias against sequences starting with uridine or adenosine (RNA templates starting with AA, AC, UG and UU were underrepresented in the sequencing reads).

FIG. 8C shows that misrepresentation of the RNA templates in a cDNA library prepared by conventional 3′ and 5′ ligation-based methods is significant. RNA templates starting with AG, GG, GU and UG were underrepresented, whereas GA was overrepresented in the sequencing reads.

FIG. 8D shows data from FIG. 8A-8C represented in a scatter dot plot format where the most accurate distribution of sequencing reads for the cDNA library was obtained from guanosine 5′-capped RNA templates (open circles). All RNA templates were expected to have a normalized reads value of 1 (solid line). The interval of 2-fold from the expected value (equivalent to a 2-fold over- or under-representation) is shown with dashed lines. Data represent mean of n=4 independent experiments.

FIG. 9 shows in a scatter plot of sequencing reads that relatively uniform reads were obtained using 16 discrete RNA templates that varied by number of phosphates between the G and the first nucleotide on the template and by replacing G with Inosine (I) in the cap.

Chemical capping was performed prior to TS to generate 5′-XpₘpNN RNA oligonucleotides, wherein the sequence ID “NN” represents the first two variable nucleotides at the 5′ end of the 25mer sequence; X is inosine (I) or guanosine (G); and m=1, 2, or 3 is the number of phosphates added through the chemical capping reaction.

A scatter dot plot distribution of the sequencing reads of cDNA libraries confirmed that unmethylated guanosine cap forming a 5′-5′ triphosphate linkage with the RNA template (5′-GpppNN) provided the most uniform and accurate distribution of sequencing reads (open circles). All RNA templates were expected to have a normalized reads value of 1 (solid line). The interval of 2-fold from the expected value (equivalent to a 2-fold over- or under-representation) is shown with dashed lines. Data represent mean of n=4 independent experiments.

FIG. 10A-10B shows that a commercially available equimolar pool of 962 unique miRNAs (miRXplore, Miltenyi Biotec, Bergisch Gladbach, Germany) that had been capped at the 5′ end (5′-Gppp) for TS and having a 3′ adaptor of known sequence added by splint ligation (3′-Splint adaptor) provided a much less biased representation of cDNA sequences than that of observed with a commercially available kit (Illumina TruSeq® Small RNA Kit (Illumina, San Diego, Calif.)).

FIG. 10A shows the distribution of sequences of the cDNA libraries made through TS of guanosine 5′-capped (5′-Gppp) miRNAs and different 3′-Adaptor ligation strategies.

FIG. 10B shows 10 randomly chosen sequences from the miRXplore cDNA library in FIG. 10A. A large number of miRNAs are underrepresented using the Illumina TruSeq Small RNA Library Prep Kit. The results demonstrate that sequencing reads originating from guanosine 5′-capped miRNAs are less biased (45%-78% of the sequencing reads were within an interval of 2-fold from the expected value) than those obtained from a conventional small RNA library preparation method (only 22% of the sequencing reads were within the 2-fold interval with the Illumina TruSeq Small RNA Library Prep Kit).

FIG. 11 shows that the chemical capping of RNA having a single phosphate at the 5′ terminus prior to capping in combination with the use of TS for adding a 5′ TSO and ligation to add a 3′ adaptor enables accurate quantification of individual RNA templates. Here human brain total RNA was spiked with varying amounts of 12 microRNAs. The sequencing reads show that the relative representation of each of the 12 microRNAs identified from the total RNA mix matched the input ratios.

FIG. 12A-12C shows that naturally occurring miRNAs can be identified using deep sequencing in complex samples consisting of total RNA extracted from different cell types after chemical capping and TS to create cDNAs.

FIG. 12A shows cDNA corresponding to miRNA in Human Brain Total RNA.

FIG. 12B shows cDNA corresponding to miRNA in Human Heart Total RNA.

FIG. 12C: shows cDNA corresponding to miRNA in Human Liver Formalin Fixed Paraffin Embedded (FFPE) Total RNA.

cDNA libraries were made through TS of guanosine 5′-capped (5′-Gppp) miRNAs and various 3′-Adaptor ligation strategies. Data represent mean of n=2 independent experiments.

FIG. 13A-13C shows that chemically capped ssDNA (5′-Gppp) can be efficiently detected by TS with less bias than uncapped ssDNA.

FIG. 13A shows capillary electrophoresis results for guanosine 5′-capped ssDNA templates from which are generated cDNA libraries with adaptors at the 5′ end introduced by TS. TS is more efficient with 5′-capped ssDNA templates than with uncapped DNA templates.

FIG. 13B shows a scatter dot plot distribution of the sequencing reads originating from cDNA libraries made from a pool of 16 discrete ssDNA templates (sequences are shown in the inset, wherein d represents deoxyribonucleotide). ssDNA templates that had been capped at the 5′ end (5′-Gppp) for TS and having a 3′ adaptor of known sequence added by splint ligation (3′-Splint adaptor) provided a less biased representation of the DNA sequences (data represented by filled circles) than observed with uncapped (5′-p) DNA templates (data represented by open squares). All DNA templates were expected to have a normalized reads value of 1 (solid line). The interval of 2-fold from the expected value (equivalent to a 2-fold over- or under-representation) is shown with dashed lines. Data represent mean of n=2 independent experiments.

### DETAILED DESCRIPTION

Methods and compositions for chemically capping polynucleotides are described herein.

Aspects of the present disclosure can be further understood in light of the embodiments, section headings, figures, descriptions and examples, none of which should be construed as limiting the entire scope of the present disclosure in any way. Accordingly, the claims set forth below should be construed in view of the full breadth and spirit of the disclosure.

Each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

Unless defined otherwise, all technical and scientific terms used herein have the same meaning as commonly understood by one of ordinary skill in the art to which this invention belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular Biology, 2d ed., John Wiley and Sons, New York (1994), and Hale & Markham, The Harper Collins Dictionary of Biology, Harper Perennial, N.Y. (1991) provide one of skill with the general meaning of many of the terms used herein.

All references cited herein are incorporated by reference.

As used herein and in the appended claims, the singular forms “a” and “an” include plural referents unless the context clearly dictates otherwise. For example, the term “a protein” refers to one or more proteins, i.e., a single protein and multiple proteins. It is further noted that the claims can be drafted to exclude any optional element. As such, this statement is intended to serve as antecedent basis for use of such exclusive terminology as “solely,” “only” and the like in connection with the recitation of claim elements or use of a “negative” limitation.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified.

Compositions and methods are provided for the preparation of cDNA libraries for sequencing including gene expression profiling, and other uses. Sequencing, including Next Generation sequencing (NGS), is a powerful tool for the analysis of cDNA libraries derived from polynucleotides as it enables the detection of single base differences between molecules, the discovery of unknown molecules and the determination of the differences in composition or expression between different samples. Polynucleotide libraries include RNA libraries. RNA libraries are typically constructed using ligation such as described for example in Hafner et al. (2008) Methods 44: 3-12]. However, these libraries are known to suffer from bias arising from the terminal RNA sequences that appear to determine ligation efficiency (Jackson, et al. (2014) BMC Genomics 15: 569; Hafner, et al. (2011) RNA 17: 1697-1712; Coenen-Stass, et al. (2018) RNA Biol. 15: 1133-1145). Another method of constructing RNA libraries uses TS but published methods also have problems of bias by Coenen-Stass, et al. (2018) RNA Biology 15: 1133-1145.

“Bias” as used herein refers to prejudice in favor of or against a polynucleotide sequence or a species of polynucleotide molecule in a mixed population of molecules. For example, bias is observed when input RNA abundance is misrepresented as determined by analysis of sequence read frequencies.

“Efficiency” as used herein refers a measure of the success of a conversion of a molecule from one form to another, for example, the success of converting an uncapped RNA into a capped RNA and/or success of the conversion of an RNA molecule into a cDNA molecule and/or the success of converting a reverse transcribed polynucleotide into one that has an adapter attached by TS.

“Population of polynucleotides” as used herein refers to multiple different polynucleotides preferably naturally occurring where the different polynucleotides may vary by their length, 5′ terminal nucleotides, and/or the presence of no 5′ phosphate or one or two 5′ phosphates. The population of polynucleotides may further include different classes of polynucleotide such as ssDNA, partially ssDNA, RNAs of different types and fragments of either RNA or DNA. The population of polynucleotides may include differing amounts of representative types of polynucleotide with very low concentrations of some of the types and more abundant representation of others. In any embodiment, a population of polynucleotides can be naturally occurring, i.e., obtained from a cell. In these embodiments, the population of polynucleotides may comprise mRNA, miRNA, fragmented RNA, RNA that has been enriched for one or more species, or RNA that has been depleted for one or more species, for example. The RNA used in the method can be from any source, e.g., a prokaryote (e.g., a bacteria) or a eukaryote (e.g., a plant or animal such as a mammal or human).

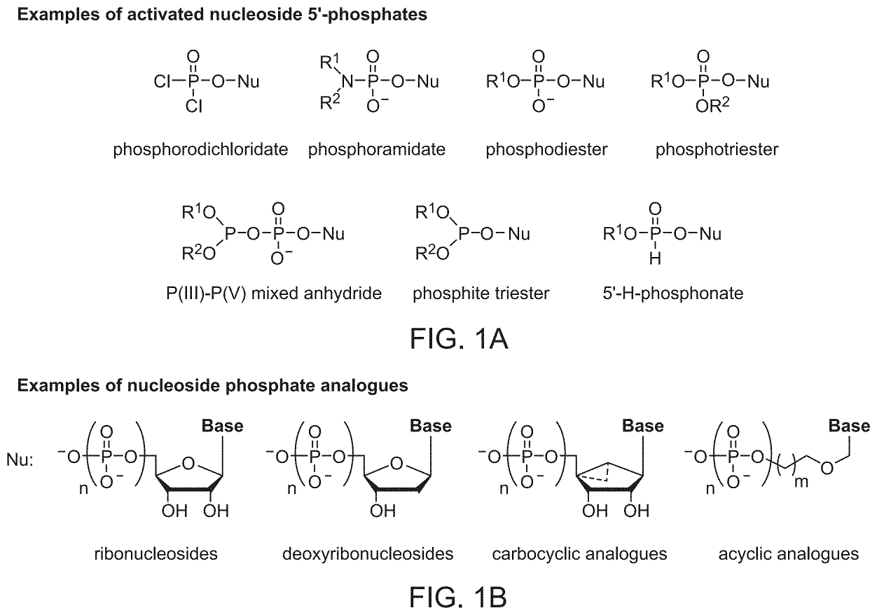

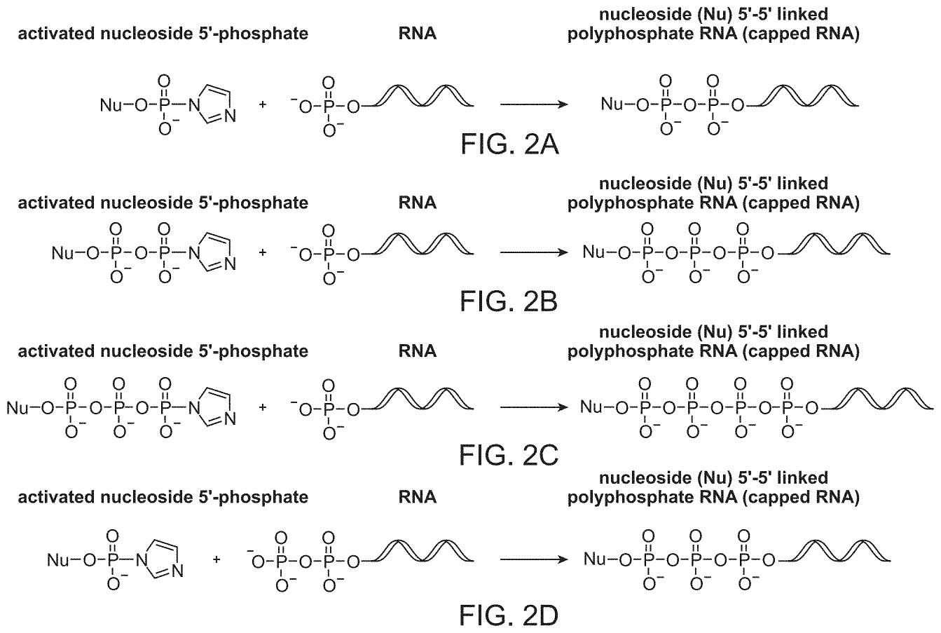

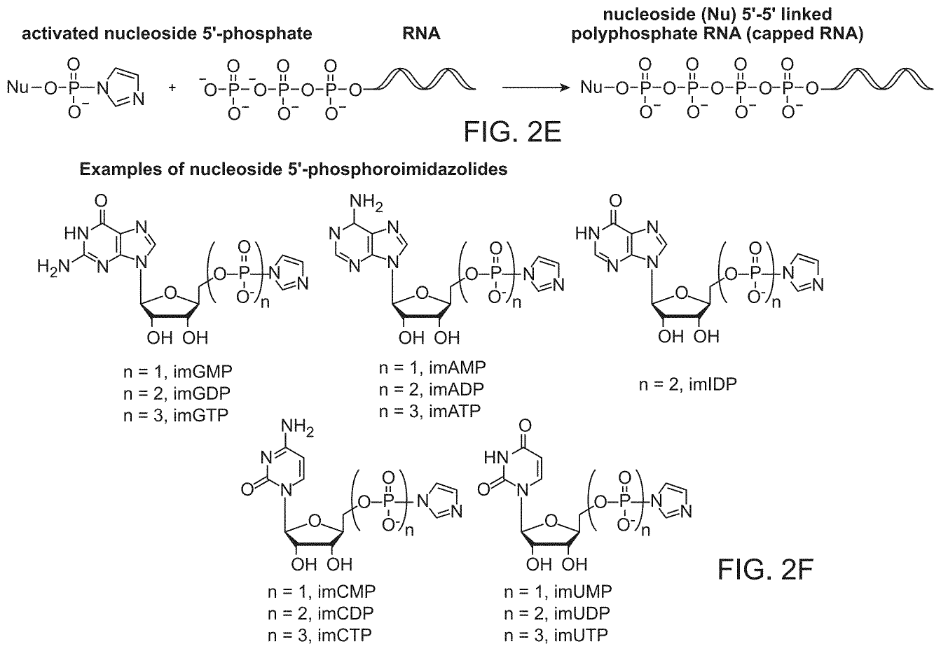

The terms “5′ cap precursor”, “activated nucleoside 5′ mono- or poly-phosphate”, and “activated nucleoside 5′ phosphate” are used interchangeably to refer to the same type of molecule. Such molecules have three components: a nucleoside, a phosphate-reactive leaving group and one or more phosphates (typically, one two or three phosphates), where the one or more phosphates join the nucleoside to the leaving group, as depicted in FIG. 1A-1B and FIG. 2A-2F. Details and examples of the components of these molecules are described in greater detail below.

The term “chemically capping” refers to a reaction that is not enzymatically catalyzed, i.e., not done using a capping enzyme. Chemical capping involves a nucleophilic substitution reaction in which the leaving group of an activated nucleoside 5′ mono- or poly-phosphate reacts with the 5′-phosphate of the polynucleotide, thereby releasing the leaving group and coupling the nucleoside 5′ mono- or poly-phosphate to the polynucleotide via a 5′ to 5′ polyphosphate linkage that comprises, e.g., 2, 3, or 4 phosphates).

The term “incubating” refers to maintaining a reaction under conditions that are suitable for production of a desired product. In the present case, the term incubating refers a reaction that occurs at a temperature of at least room temperature (e.g., room temperature to about 60° C.) for at least 30 minutes (e.g., 1 hour to 10 hours or 1-6 hours).

The term “template-switching” (TS) reaction refers to the template-dependent addition the complement of an oligonucleotide (referred to as a template-switching oligonucleotide or TSO) to the 3′ end of a cDNA during cDNA synthesis. TS is believed to be caused by a RT switching template from one molecule (an RNA) to another (the TSO) during cDNA synthesis. TS and TSOs are described in, e.g., Luo, et. al., (J. Virol., 1990 64: 4321-4328) and Zhu, et al. (Biotechniques 2001 30: 892-897). In TS, reverse transcription is done in the presence of the TSO.

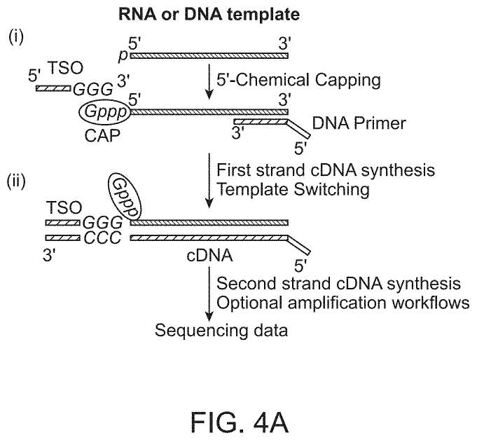

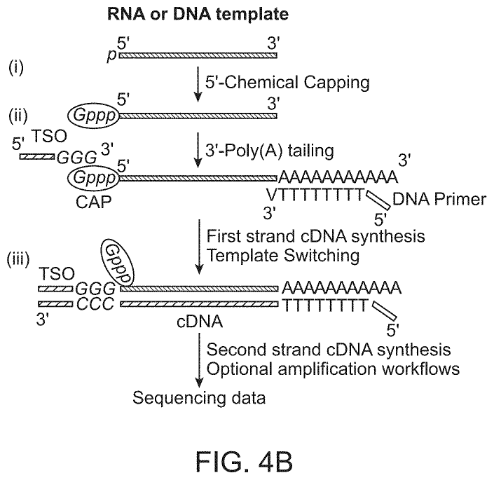

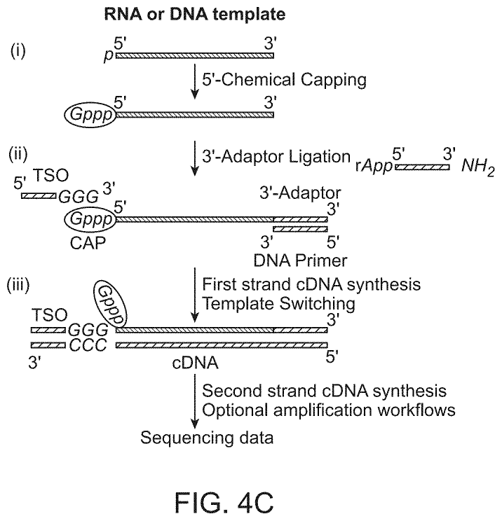

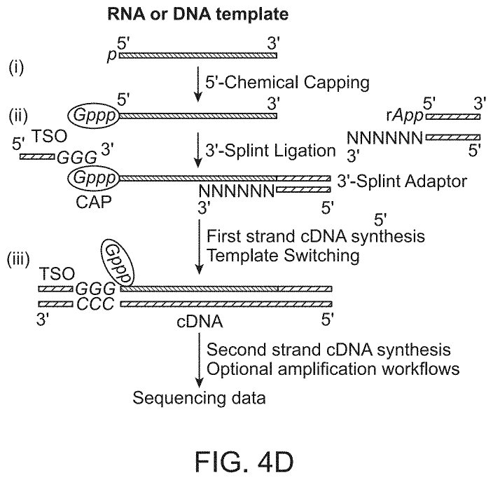

The term “reverse transcribing” refers to a template-dependent reaction in which an RNA dependent DNA polymerase (an RT) copies RNA into DNA (i.e., cDNA). As illustrated in FIG. 4A-4D, RNA can be copied into cDNA using one or more gene-specific primers, random primers or an oligo dT primer (in which case the 3′ end of the RNA does not need to be modified before reverse transcription). RNA can also be copied into cDNA using a universal primer (in which case an adapter that provides a binding site for the universal primer can be added to the 3′ end of the RNA beforehand). In some embodiments, the RNA may be polyA tailed using a polyA polymerase, and the polyA tailed RNA may be copied into cDNA using an oligo dT primer. Although the term “reverse transcribing” is commonly understood as RNA being copied into cDNA, herein the same term is broadly used and refers to any polynucleotide (including RNA, DNA, or hybrids thereof) being copied into cDNA.

A solution to the problem of bias and efficiency in library preparation is provided here by chemically capping DNA or RNA molecules in a population of polynucleotides and template switching reverse transcription. A 5′ cap precursor is added to the 5′ end of a polynucleotide molecule having a 5′phosphate in a population of polynucleotides to form 5′ capped polynucleotides through a 5′ to 5′ polyphosphate covalent linkage. An RT then generates a cDNA and continues on to add an adapter sequence that is complementary to a TSO.

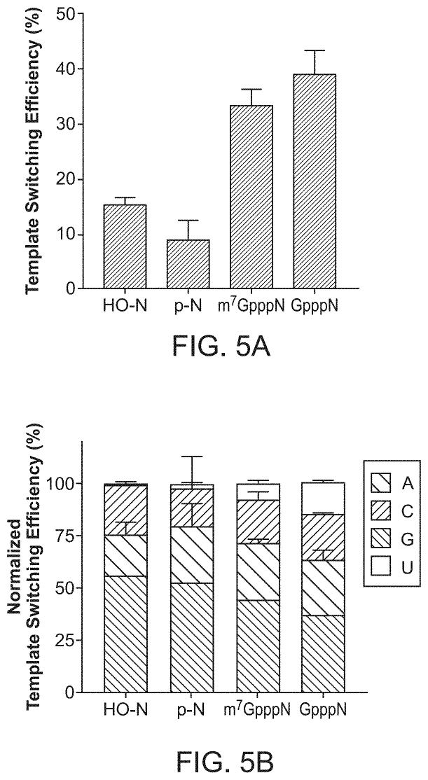

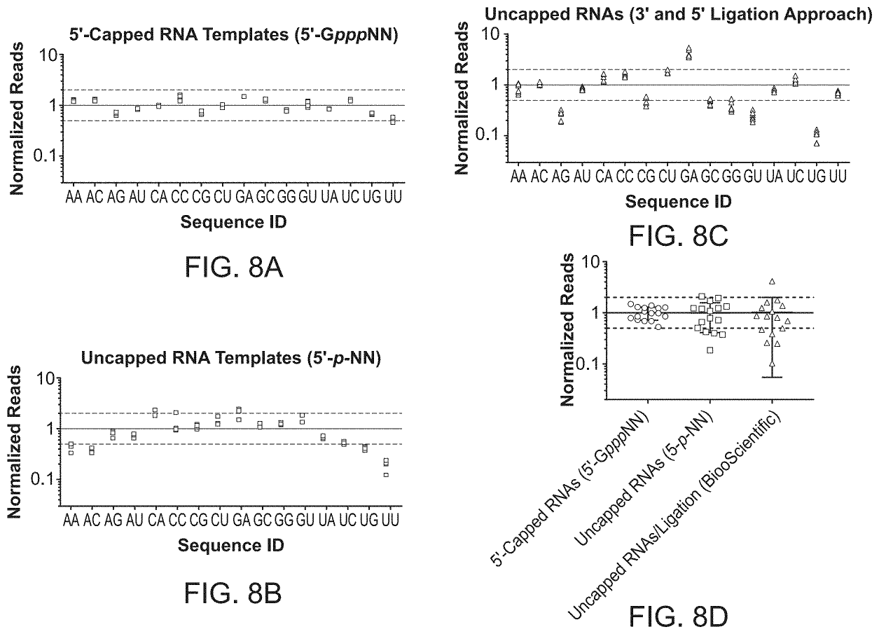

Advantages of this approach compared to other methods of generating cDNAs include one or more of the following:
a) Improvement in capping efficiency: Capped polynucleotides promote TS more efficiently than uncapped polynucleotides, including DNA and RNA; (see for example FIG. 5A-5B, FIG. 8A-8D, and FIG. 13A-13C);
b) Greater yields and less bias in sequencing libraries: Guanosine (G) cap showed higher yield and less bias than does 7-Methylguanosine (m⁷G) cap. (see for example, FIG. 5A-5B); and
c) Greater efficiency and reduced bias for cDNA from damaged RNA, non-capped RNA and ssDNA: Chemical capping improves efficiency and reduce sequencing biases of damaged RNA (see for example FIG. 12C), non-capped RNA (see for example, small RNAs; as shown in FIG. 10A-10B, FIG. 11 and FIG. 12A-12C), and ssDNA (see for Example, FIG. 13A-13C).

Through increased yield of cDNA, reduced biases of varying sequences of polynucleotides in the population and improved accuracy of cDNA libraries that represent the varied polynucleotides from the sample, the method enables the analysis of small amounts of polynucleotide in a population where the population of polynucleotides from the sample may include uncapped RNAs as well as degraded and/or fragmented RNAs and DNAs.

### Chemical Capping

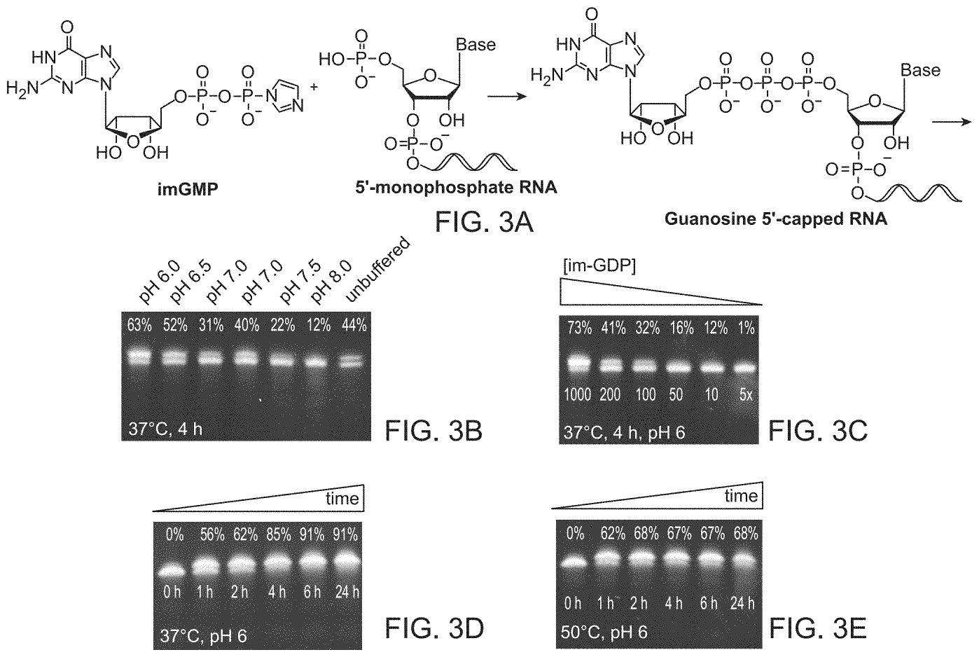

The compositions and methods described below demonstrate high efficiency and relatively rapid production of capped polynucleotides. The efficiency of capping compared to previously described capping, increased by more than 1.5-fold within a time period of less than 10 hours, more particularly less than 6 hours, more particularly 5 hours or less (see for example FIG. 3B-3E).

The above results demonstrate a significant improvement over the non-enzymatic capping of 5′-monophosphate oligonucleotides with 7-methylated guanosine described by Sawai, et al. J. Org. Chem. 64, 5836-5840 (1999) which reported low to moderate yields of the capping reaction (35%-49%) with incubation times of 4 to 10 days that limited the practicability of this capping method. These improvements for the first time enable capping of polynucleotide molecules from complex samples such as cell extracts or formalin-fixed, paraffin-embedded tissue.

Chemical capping as described herein was used to attach a synthetic 5′ cap structure (including guanosine, adenosine, cytidine, inosine and uridine caps as well as di-, tri and tetraphosphate bridges) to uncapped nucleic acids to generate polynucleotide sequencing libraries. The preferred cap was found to be unmethylated guanosine caps unknown in stable RNAs in nature. An unmethylated guanosine 5′ cap produced a more efficient and less sequence biased template switch than was observed for m⁷G caps that are found in nature on intact mRNAs.

Chemical capping was carried out in the presence of activated 5′-nucleotide precursors. The synthesis of activated 5′-nucleotides was based on the nucleophilic substitution of a nucleoside or nucleoside 5′-phosphate, such as a nucleoside 5′-monophosphate, NMP; a nucleoside 5′-diphosphate, NDPs; and a nucleoside 5′-triphosphate, NTP. Nucleosides and nucleoside 5′-phosphates can be activated as a phosphorodichloridate, phosphoramidate, phosphodiester, phosphotriester, 5′-H-phosphonate, P(III)—P(V) mixed anhydride, or phosphite triester (FIG. 1A). P(III) and P(V) activation chemistries (reviewed by Roy, et al. Chem. Rev., 116, 7854-7897 (2016)) may be applied to a large number of nucleoside 5′-phosphate analogues, including ribonucleosides or deoxyribonucleosides, base- or sugar-modified nucleosides, as well as carbocyclic and acyclic analogues (FIG. 1B).

The chemistry for synthesizing nucleoside 5′-phosphate precursors is described in more detail below.

P(III) and P(V) activation chemistries may be used to produce analogues containing phosphate modifications, such as thiophosphates, boranophosphates, and selenophosphates. Nucleosides 5′-phosphates, as well as their carbocyclic and acyclic analogues, activated as any of the reagents described above can be used in coupling reactions with an oligonucleotide 5′-phosphate (e.g., an oligonucleotide 5′-monophosphate, 5′-diphosphate, or 5′-triphosphate) to form products comprising 5′ to 5′ polyphosphate linkages. The coupling reaction with an oligonucleotide 5′-phosphate may be performed in an aqueous solvent, an organic solvent, or a combination thereof; it may include an inorganic metal salt as a catalyst; and it may also include further additives such as polyethylene glycols (PEGs) and PEG derivatives. Phosphorodichloridates can be generated for example by the reaction of nucleosides with phosphoryl chloride in trimethylphosphate.

Nucleoside 5′-phosphodiesterscan be generated for example by the reaction of 2′,3′-protected nucleosides with 2-cyanoethyl phosphate in the presence of N,N′-dicyclohexyl carbodiimide (DCC), followed by in situ removal of the cyanoethyl group. Another example of a phosphorylating reagent is the 2-O-(4,4′-dimethoxytrityl)ethylsulfonylethan-2′-yl-phosphate, which forms a phosphodiester intermediate in the presence of a suitably protected nucleoside and triisopropylbenzenesulfonyl tetrazolide (TPSTAZ).

Nucleoside 5′-phosphotriesters can be generated for example by a Mitsunobu-type coupling reaction between a nucleoside and a dibenzyl phosphate in the presence of triphenylphosphine and diethylazodicarboxylate; subsequent debenzylation produces the phosphotriester intermediate. Another approach to a phosphotriester involves the reaction of suitably protected nucleosides first with di-tert-butyloxy N,N-diethylphosphoramidite in the presence of 1H-tetrazole and then oxidation with meta-chloroperoxybenzoic acid (m-CPBA); subsequent removal of tert-butyl and acetonide groups produces the phosphotriester intermediate. A further approach to a phosphotriester makes use of salicylic alcohols to mask the phosphate group (cycloSal phosphate). CycloSal phosphotriesters can be synthesized via P(III) and P(V) chemistries. One approach to cycloSal phosphotriesters via P(III) is based on the coupling of a nucleoside with saligenylchlorophosphite, followed by in situ oxidation. A second approach to cycloSal phosphotriesters via P(III) involves the reaction of a nucleoside with a phosphoramidite and then the oxidation of the phosphite trimester. A third approach to cycloSal phosphotriesters via P(III) involves the reaction of a nucleoside with a cyclosaligenylphosphorochloridate. A fourth approach to cycloSal phosphotriesters via P(III) involves the prior synthesis of nucleoside phosphorodichloridate, which is then treated with salicylic alcohol.

Nucleoside 5′-H-phosphonates can be prepared for example through the transesterification of diphenyl H-phosphonate with suitably protected nucleosides in pyridine to produce phenyl H-phosphonate diesters; subsequent treatment with aqueous triethylamine produces the H-phosphonate monoester intermediate. H-phosphonate monoesters may be converted into trivalent silyl phosphites, for example, by treatment with N,O-bis(trimethylsilyl) acetamide (BSA), to facilitate further oxidation to the corresponding phosphates.

Nucleosides comprising P(III)—P(V) mixed anhydrides can be generated for example by phosphitylation of nucleoside 5′-monophosphates in the form of their tetra-N-butyl or tris-N-hexyl ammonium salts with a phosphoramidite reagent (e.g., salicylic chlorophosphite or bis-diisopropylamino chlorophosphine) followed by oxidation with an aqueous pyridine solution of iodine. Suitably protected nucleosides may be required in this approach to prevent any side reactions; however, the use of bulky phosphitylation reagents enable the reaction with unprotected nucleosides. P(III)—P(V) mixed anhydrides can be used to produce analogues containing modifications at the α-phosphate, such as 5′-(α-P-thiophosphates), 5′-(α-P-boranophosphates), and 5′-(α-P-selenophosphates).

Nucleoside 5′-phosphosulfonyl reagents can be generated for example by reacting tetra n-butylammonium salts of nucleoside 5-phosphates with a sulfonylimidazolium salt in the presence of N-methylimidazole (NMI) or N,N′-diisopropylethylamine (DIPEA) as a base. The sulfonylimidazolium salt can prepared by reacting phenylsulfonylimidazolide with methyl triflate in ether.

Nucleoside 5′-phosphoramidates are some of the most useful precursors for the synthesis of phosphate-phosphate linkages. Examples of phosphoramidates include phosphoroimidazolides, phosphoromorpholidates, phosphoropiperidates, pyrrolidinium phosphoramidates and pyridinium phosphoramidates.

Nucleoside 5′-phosphoropiperidates can be generated for example by phosphitylation of carboxybenzyl-protected nucleosides with benzyl N,N-diisopropylchlorophosphoramidite in the presence of 1H-tetrazole, and then oxidative coupling with CCl₄/Et₃N/piperidine; subsequent deprotection of carboxybenzyl and benzyl esters of the nucleosidic and phosphoramidate moieties by mild catalytic hydrogenation produces the phosphoropiperidate. The coupling of 5′-phosphoropiperidates with phosphate-containing compounds to form phosphate-phosphate linkages can be promoted by 4,5-dicyanoimidazole (DCI).

Nucleoside 5′-phosphoromorpholidates can be generated for example by reacting a nucleoside or a nucleoside 5′-phosphate with 2,2,2-tribromoethyl phosphoromorpholinochloridate followed by the in situ removal of the 2,2,2-tribromoethyl protecting group with Cu—Zn. Another approach to 5′-phosphoromorpholidates is by coupling a nucleoside 5′-phosphate with morpholine in the presence of DCC.

Nucleoside 5′-pyrrolidinium phosphoramidates can be generated for example by rearrangement of nucleoside phosphoramidate diesters derived from N-(3-chlorobutyl)-N-methylamine leading to the formation of the pyrrolidinium phosphoramidates.

Nucleoside 5′-pyridinium phosphoramidates can be generated from nucleoside 5′-H-phosphonate monoesters derived either from salicylchlorophosphite or phosphorus trichloride. Silylation of the H-phosphonate monoesters with TMSCl in pyridine, followed by oxidation with iodine result in the corresponding pyridinium phosphoramidites. Nucleoside 5′-phosphoroimidazolides are preferred reagents as they are more reactive than the corresponding phosphoromorpholidates, and more permissive when it comes to the choice of solvent. Phosphoroimidazolides are also sometimes referred as phosphoroimidazolates, phosphoroimidazolidates, phosphoroimidazoles, phosphorimidazolidates, phosphorimidazolates, phosphorimidazolides, and phosphorimidazoles.

Nucleoside 5′-phosphoroimidazolides can be prepared for example by treatment of nucleoside 5′-phosphates (including nucleoside 5′-monophosphates, NMPs; nucleoside 5′-diphosphates, NDPs; nucleoside 5′-triphosphates, NTPs; nucleoside 5′-tetraphosphates; nucleoside 5′-pentaphosphates; nucleoside 5′-hexaphosphates; and so forth) with 1,1′-carbonyldiimidazole (CDI), followed by removal of the 2′,3′-carbonate protecting group under basic conditions. Another strategy to prepare phosphoroimidazolides is by treatment of nucleoside 5′-phosphates with imidazole in the presence of triphenylphosphine and 2,2′-dithiodipyridine. The latter strategy can also be performed with imidazole derivatives such as N-methylimidazole, 2-methylimidazole, 4-methylimidazole, 2-aminoimidazole, 2-isopropylimidazole, 2-phenylimidazole, benzimidazole, 2-methylbenzimidazole, 2-chloroimidazole, or 2-methylaminoimidazole. Yet another strategy to prepare phosphoroimidazolides is by in situ activation of nucleoside 5′-phosphates with trifluoroacetic anhydride (TFAA) in the presence of a tertiary amine in acetonitrile, followed by removal of excess TFAA under vacuum and treatment of the resulting mixed anhydrides with N-methylimidazole to produce the corresponding phosphoromethylimidazolides.

Phosphoroimidazolide activation chemistry may be applied to a large number of nucleoside 5′-phosphate analogues, including ribonucleosides or deoxyribonucleosides, base- or sugar-modified nucleosides, as well as carbocyclic and acyclic analogues. The resulting nucleoside 5′-phosphoroimidazolides can be isolated by precipitation for example by treatment with sodium or lithium perchlorate in acetone followed by filtration.

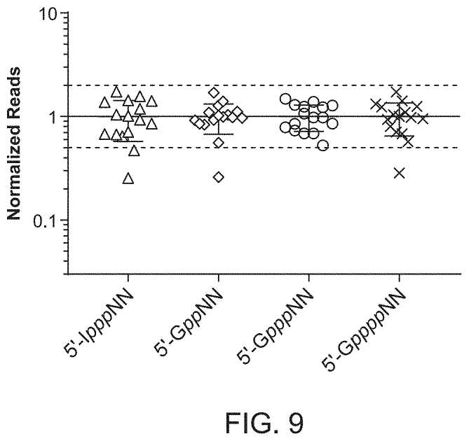

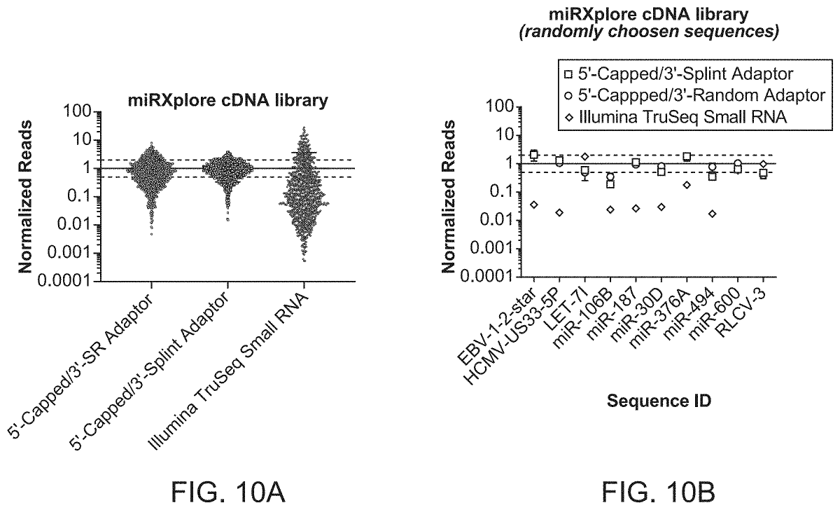

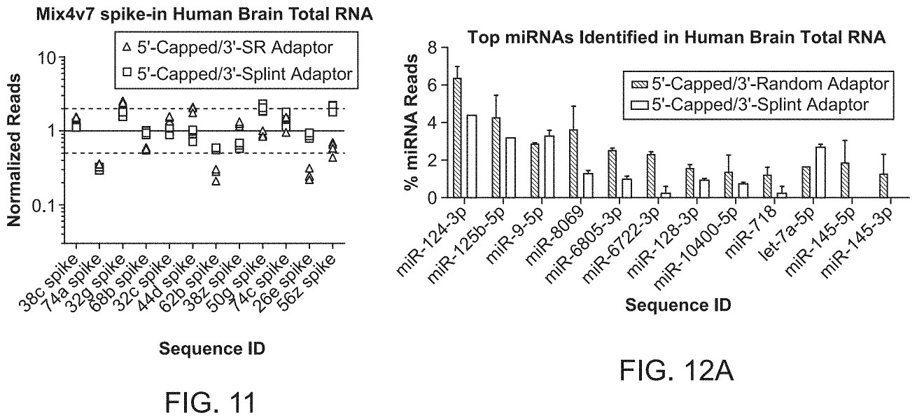

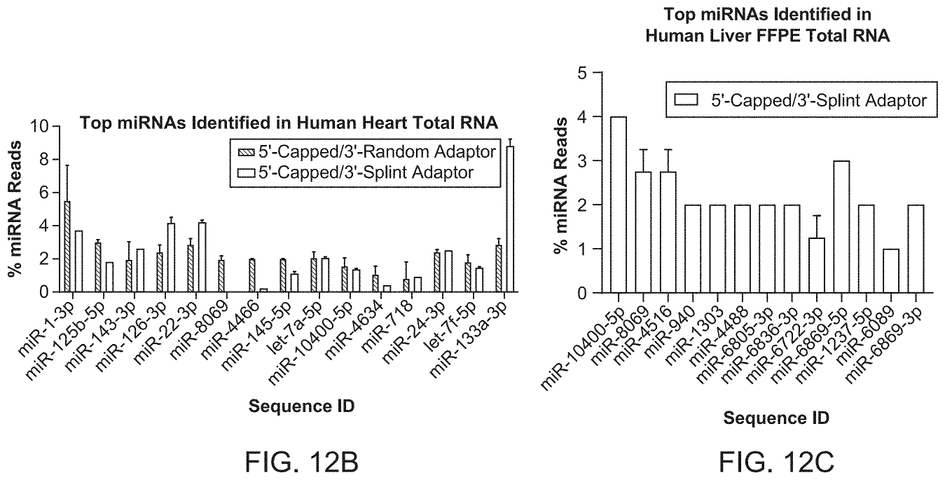

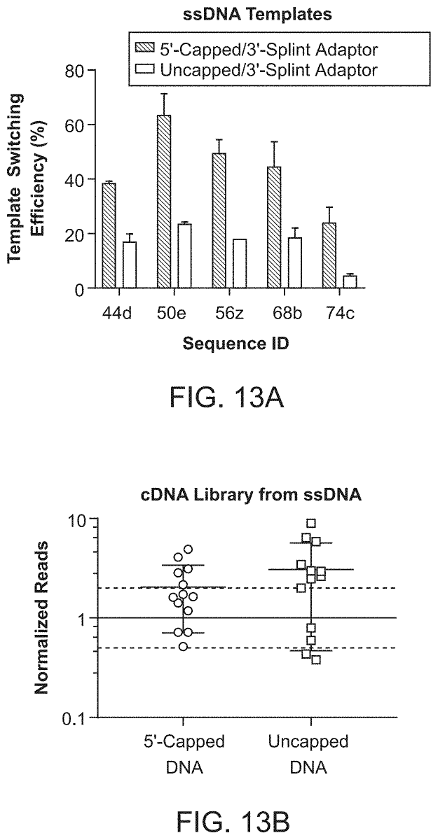

The nucleoside 5′-phosphoroimidazolide can be derived from a guanosine phosphate, an adenosine phosphate, a cytidine phosphate, or an inosine phosphate where the phosphate may be a monophosphate, diphosphate, triphosphate or tetraphosphate that after chemical capping will result in a polynucleotide with a 5′-5′ di-, tri- or tetra- or penta-phosphate linked nucleoside cap (see for example FIG. 2A-2F, FIG. 9 and Example 3).

The 2′-position of the nucleoside 5′-phosphate may be an SH, NH₂, a lower alkyl (e.g., methyl), a lower alkoxy (e.g., methoxy), a lower acyloxy (e.g., acetoxy), a lower alkylamine (e.g., methylamine), a lower acylamine (e.g., acetamide), halogenyl, allyl, propargyl, or N₃. In some embodiments, the 3′-position of the nucleoside 5′-phosphate is SH, NH₂, a lower alkyl (e.g., methyl), a lower alkoxy (e.g., methoxy), a lower acyloxy (e.g., acetoxy), a lower alkylamine (e.g., methylamine), a lower acylamine (e.g., acetamide), halogenyl, allyl, propargyl, or N₃. In some embodiments, both 2′- and 3′-positions of the nucleoside 5′-phosphate are modified with the same or different groups above mentioned. In some embodiments, the nucleoside 5′-phosphate further comprises one or more fluorescent, quencher or affinity groups attached either at the nucleobase or at the 2′- or 3′-position.

Any of the chemistries described above may be applied to the synthesis of a large number of nucleoside 5′-phosphate analogues, including ribonucleosides or deoxyribonucleosides, base- or sugar-modified nucleosides, as well as carbocyclic and acyclic analogues. Examples of base modifications include, but are not limited to, those found in 2-aminopurine, 2,6-diaminopurine, 5-iodouracil, 5-bromouracil, 5-fluorouracil, 5-hydroxyuracil, 5-hydroxymethyluracil, 5-formyluracil, 5-proprynyluracil, 5-methylcytosine, 5-hydromethylcytosine, 5-formylcytosine, 5-carboxycytosine, 5-iodocytosine, 5-bromocytosine, 5-fluorocytosine, 5-proprynylcytosine, 4-ethylcytosine, 5-methylisocytosine, 5-hydroxycytosine, 4-methylthymine, thymine glycol, ferrocene thymine, pyrrolo cytosine, inosine, 1-methyl-inosine, 2-methylinosine, 5-hydroxybutynl-2′-deoxyuracil (Super T), 8-aza-7-deazaguanine (Super G), 5-nitroindole, formylindole, isothymine, isoguanine, isocytosine, pseudouracil, 1-methyl-pseudouracil, 5,6-dihydrouracil, 5,6-dihydrothymine, 7-methylguanine, 2-methylguanine, 2,2-dimethylguanine, 2,2,7-trimethylguanine, 1-methylguanine, hypoxanthine, xanthine, 2-amino-6-(2-thienyl)purine, pyrrole-2-carbaldehyde, 4-thiouracil, 4-thiothymine, 2-thiothymine, 5-(3-aminoallyl)-uracil, 5-(carboxy)vinyl-uracil, 5-(1-pyrenylethynyl)-uracil, 5-fluoro-4-O-TMP-uracil, 5-(C2-EDTA)-uracil, C4-(1,2,4-triazol-1-yl)-uracil, 1-methyladenine, 6-methyladenine, 6-thioguanine, thienoguanine, thienouracil, thienocytosine, 7-deaza-guanine or adenine, 8-amino-guanine or adenine, 8-oxo-guanine or adenine, 8-bromo-guanine or adenine, ethenoadenine, 6-methylguanine, 6-phenylguanine, nebularine, pyrrolidine, and puromycin. Examples of sugar modifications include but are not limited to those found in dideoxynucleotides (e.g., ddGTP, ddATP, ddTTP, and ddCTP), 2′- or 3′-O-alkyl-nucleotides (e.g., 2′-O-methyl-nucleotides and 3′-O-methyl-nucleotides), 2′- or 3′-O-methoxyethyl-nucleotides (MOE), 2′- or 3′-fluoro-nucleotides, 2′- or 3′-O-allyl-nucleotides, 2′- or 3′-O-propargyl-nucleotides, 2′- or 3′-amine-nucleotides (e.g., 3′-deoxy-3′-amine-nucleotides), 2′- or 3′-O-alkylamine-nucleotides (e.g., 2′-O-ethylamine-nucleotides), 2′- or 3′-O-cyanoethyl-nucleotides, 2′- or 3′-O-acetalester-nucleotides, 4′-C-aminomethyl-2′-O-methyl-nucleotides, and 2′- or 3′-azido-nucleotides (e.g., 3′-deoxy-3′-azide-nucleotides). Further examples of sugar modifications include those found in the monomers that comprise the backbone of synthetic nucleic acids such as 2′-O,4′-C-methylene-β-D-ribonucleic acids or locked nucleic acids (LNAs), methylene-cLNA, 2′,4′-(N-methoxy)aminomethylene bridged nucleic acids (N-MeO-amino BNA), 2′,4′-aminooxymethylene bridged nucleic acids (N-Me-aminooxy BNA), 2′-O,4′-C-aminomethylene bridged nucleic acids (2′,4′-BNA(NC)), 2′4′-C—(N-methylaminomethylene) bridged nucleic acids (2′,4′-BNA(NC)[NMe]), peptide nucleic acids (PNA), triazole nucleic acids, morpholine nucleic acids, amide-linked nucleic acids, 1,5-anhydrohexitol nucleic acids (HNA), cyclohexenyl nucleic acids (CeNA), arabinose nucleic acids (ANA), 2′-fluoro-arabinose nucleic acids (FANA), α-L-threofuranosyl nucleic acids (TNA), 4′-thioribose nucleic acids (4′S-RNA), 2′-fluoro-4′-thioarabinose nucleic acids (4′S-FANA), 4′-selenoribose nucleic acids (4′Se-RNA), oxepane nucleic acids (ONA), and methanocarba nucleic acids (MC).

Nucleoside 5′-phosphoroimidazolides may be used to form 5′-capped polynucleotides with phosphate modifications, such as phosphororothioates (replacement of one non-bridging oxygen atom of the phosphate group with a sulfur atom), phosphorodithioates (both non-bridging oxygen atoms of the phosphate group are replaced with sulfur), alkyphosphonates (a non-bridging oxygen atom of the phosphate group has been replaced with alkyl group, e.g. methyl), arylphosphonates (a non-bridging oxygen atom of the phosphate group has been replaced with aryl group, e.g. phenyl), N-phosphoramidates (an oxygen atom is replaced with an amino group either at the 3′- or 5′-oxygen), boranophosphates (one non-bridging oxygen atom of the phosphate group is replaced with BH3), phosphonoacetates (PACE, one non-bridging oxygen atom of the phosphate group is replaced with an acetate group), and 2′,5′-phosphodiester linkages.

Nucleoside 5′-phosphoroimidazolides may be used to form 5′-capped polynucleotides with one or more fluorescent or quencher groups, affinity groups (e.g., biotin, desthiobiotin, digoxigenin, glutathione, heparin, maltose, coenzyme A, poly-histidine, and others), haptens to an antibody (e.g., HA-tag, c-myc tag, FLAG-tag, S-tag, among many others), mono- or oligosaccharide ligands to a lectin, hormones, cytokines, toxins, and vitamins. Examples of specific binding partners to the aforementioned affinity groups, in no particular order, include but are not limited to avidin, streptavidin, neutravidin, maltose-binding protein, glutathione-S-transferase (GST), antibodies, lectins, nickel, cobalt, zinc, and poly-histidine. Further examples include groups that form an irreversible bond with a protein tag, including benzylguanine or benzylchoropyrimidine (SNAP-Tag® (New England Biolabs, Ipswich, Mass.)); benzylcytosine (CLIP-Tag™ (New England Biolabs, Ipswich, Mass.)); haloalkane (HaloTag® (Promega, Madison, Wis.)); CoA analogues (MCP-tag and ACP-tag); trimpethoprim or methotrexate (TMP-tag); FlAsH or ReAsH (Tetracysteine tag); a substrate of biotin ligase; a substrate of phosphopantetheline transferase; and a substrate of lipoic acid ligase. An affinity group is used for selectively enriching samples by means of affinity purification methods, wherein the affinity binding partner is immobilized in a column, bead, microtiter plate, membrane or other solid support. In some embodiments, the nucleoside 5-phosphate comprises a cleavable linker between the affinity group and the site of attachment to the nucleoside 5-phosphate. This strategy allows specific elution of target of interest. The cleavable linker can be cleavable, for example, by chemical, thermal or photochemical reaction. Chemically cleavable linkers include disulfide bridges and azo compounds (cleaved by reducing agents such as dithiothreitol (DTT), β-mercaptoethanol or tris(2-carboxyethyl)phosphine (TCEP)); hydrazones and acylhydrazones (cleaved by transimination in a mildly acidic medium); levulinoyl esters (cleaved by aminolysis, e.g. by hydroxylamine or hydrazine); thioesters, thiophenylesters and vinyl sulfides (cleaved by thiol nucleophiles such as cysteine); orthoesters, ketals, acetals, vinyl ethers, phosphoramidates and β-thiopropionates (cleaved by acidic conditions); vicinal diols (cleaved by oxidizing agents such as sodium periodate); and allyl esters, 8-hydroxyquinoline esters, and picolinate esters (cleaved by organometallic and metal catalysts). Further examples include, acid or base labile groups, including among others, diarylmethyl or trimethylarylmethyl groups, silyl ethers, carbamates, oxyesters, thiesters, thionoesters, and α-fluorinated amides and esters. Examples of photocleavable cleavable linkers include o-nitrophenyl group, diazobenzene, phenacyl, alkoxybenzoin, benzylthioether and pivaloyl glycol derivatives.

Nucleoside 5′-phosphoroimidazolides may be used to form 5′-capped polynucleotides with one or more reactive groups. In some embodiments, the analogue comprises a reactive group selected from the group consisting of a carbonyl; a carboxyl; an active ester, e.g. a succinimidyl ester; a maleimide; an amine; a thiol; an alkyne, an azide; an alkyl halide; an isocyanate; an isothiocyanate; an iodoacetamide; a 2-thiopyridine; a 3-arylproprionitrile; a diazonium salt; an alkoxyamine; a hydrazine; a hydrazide; a phosphine; an alkene; a semicarbazone; an epoxy; a phosphonate; and a tetrazine. Examples of chemoselective reactions are: between an amine reactive group and an electrophile such an alkyl halide or an N-hydroxysuccinimide ester (NHS ester); between a thiol reactive group and an iodoacetamide or a maleimide; between an azide and an alkyne (azide-alkyne cycloaddition or “Click Chemistry”). Examples and uses of chemoselective reactions in biological systems are reviewed in a variety of publications, such as in Sletten, E. M. and Bertozzi, C. R. “Bioorthogonal Chemistry: Fishing for Selectivity in a Sea of Functionality” Angewandte Chemie International Edition English 2009, 48(38): 6974-98.

### Capping Reaction

A nucleoside 5′-phosphoroimidazolide can be used in a capping reaction with a polynucleotide 5′-phosphate (e.g., a 5′-monophosphate, 5′-diphosphate, 5′-triphosphate, 5′-tetraphosphate, and so forth) to form 5′ to 5′ polyphosphate linkages (see for example, FIG. 2A-2E). The nucleoside 5′-phosphoroimidazolide may be used in a capping reaction in a molar excess from 2 to 1000000-fold relative to the polynucleotide 5′-phosphate, with 1000-fold (1000×) being the most preferred.

The coupling reaction between a nucleoside 5′-phosphoroimidazolide and a polynucleotide 5′-phosphate may be performed in an aqueous buffer, an organic solvent, or a combination thereof with a pH in the range of 4 to 7, preferably 5 to 6. An example of an aqueous buffer includes a non-nucleophilic, phosphate-free buffer, such as ADA (N-(2-Acetamido)-2-iminodiacetic acid), BES (N,N-Bis(2-hydroxyethyl)-2-aminoethanesulfonic acid), BICINE (N,N-Bis(2-hydroxyethyl)glycine), DIPSO (3-(N,N-Bis[2-hydroxyethyl]amino)-2-hydroxypropanesulfonic acid), EPPS (4-(2-Hydroxyethyl)-1-piperazinepropanesulfonic acid), HEPBS (N-(2-Hydroxyethyl)piperazine-N′-(4-butanesulfonic acid)), 4-Ethylmorpholine, MOBS (4-(N-Morpholino)butanesulfonic acid), MOPS (3-(N-Morpholino)propanesulfonic acid), MOPSO (3-(N-Morpholinyl)-2-hydroxypropanesulfonic acid), PIPES (1,4-Piperazinediethanesulfonic acid), POPSO (Piperazine-N,N′-bis(2-hydroxypropanesulfonic acid)), TEA (triethylammonia), BIS-TRIS (2,2-Bis(hydroxymethyl)-2,2′,2″-nitrilotriethanol), BIS-TRIS propane (1,3-Bis[tris(hydroxymethyl)methylamino]propane), or a combination thereof. Examples of organic solvent include: alcohols, such as ethanol, 1-propanol, 2-propanol, 1-butanol, 2-butanol, t-butyl alcohol; or nitriles, such as acetonitrile or propionitrile; amides such as N,N-dimethylformamide (DMF), N,N-dimethylacetamide (DMA), N-methyl-2-pyrrolidone; sulfoxides such as dimethylsulfoxide (DMSO); ethers such as diethyl ether, diisopropyl ether, methyl t-butyl ether, tetrahydrofuran, 1,4-dioxane, 2-methoxyethanol, anisole; or any mixtures of one or more of these solvents. A preferred coupling reaction buffer includes a 10% to 40% organic solvent in aqueous buffer solution, most preferably 20%.

The coupling reaction between a nucleoside 5′-phosphoroimidazolide and a polynucleotide 5′-phosphate may include an inorganic metal salt as a catalyst. Examples include a halide, sulphate, nitrate, phosphate, hydrogen phosphate, or hydrogen sulfate salts; wherein the inorganic metal is magnesium, manganese, zinc, or cobalt. Preferably, the inorganic metal salt is MgCl₂, MnCl₂, ZnCl₂, or CoCl₂.

The coupling reaction between a nucleoside 5′-phosphoroimidazolide and a polynucleotide 5′-phosphate may further include additives such as polyethylene glycols (PEGs) and PEG derivatives such as PEG ethers (e.g., laureths, ceteths, ceteareths, and oleths), PEG fatty acids (e.g., PEG laurates, dilaurates, stearates, and distearates), PEG amine ethers (e.g., PEG cocamines), PEG propylene glycols, or other derivates. Preferably are PEGs with molecular weight (MW) ranging from 1,000 to 20,000 Da. PEGs may comprise mixtures of different oligomer sizes.

The coupling reaction between a nucleoside 5′-phosphoroimidazolide and a polynucleotide 5′-phosphate may further include one or more surfactants. Examples of surfactants include polyoxyethanyl-alpha-tocopheryl sebacate (PTS); DL-alpha-tocopherol methoxypolyethylene glycol succinate (TPGS-750-M); beta-sitosterol methoxyethylene glycol succinate (SPGS-550-M); bis(4-((2-(Methoxycarbonyl)phenyl)amino)-4-oxobutanoic acid)polyethylene glycol 1000; and combinations thereof.

The coupling reaction between a nucleoside 5′-phosphoroimidazolide and a polynucleotide 5′-phosphate may be performed in a range of 20° C. to 70° C. The reaction times typically range from less than one hour up to 24 hours. For example, the coupling reaction temperature and time may be 50° C. and 5 hours when using 5′-phosphoroimidazolides derived from nucleoside 5′-monophosphates (imNMPs); 37° C. and 4 hours when using 5′-phosphoroimidazolides derived from nucleoside 5′-diphosphates (imNDPs); or room temperature and 4 hours when using 5′-phosphoroimidazolides derived from nucleoside 5′-triphosphates (imNTPs).

A suitable nucleoside 5′-phosphoroimidazolide and/or polynucleotide 5′-phosphate salt may be formed with a suitable cation selected from, but is not limited to: inorganic cations, such as Na⁺, K⁺, Ca⁺, Mg⁺, Mn²⁺, Zn²⁺, Co²⁺, and Al⁺³; ammonium ions (i.e., NH₄⁺); and substituted ammonium ions (e.g., NH₃R⁺, NH₂R₂⁺, NHR₃⁺, and NR₄⁺), wherein the substituted ammonium ions derives from alkyl and aryl amines such as ethylamine, diethylamine, dicyclohexylamine, triethylamine, butylamine, hexylamine, ethylenediamine, ethanolamine, diethanolamine, piperazine, pyridine, benzylamine, and any combinations thereof.

The polynucleotide 5′-phosphate may be DNA, or RNA, or a chimeric polynucleotide (chimera) consisting of RNA and DNA bases. The polynucleotide 5′-phosphate may be single-stranded, double-stranded, or consist of a chimera of partially single-stranded and double-stranded segments. The polynucleotide 5′-phosphate may also be a double-stranded segment having 3′ or 5′ end nucleotide extensions. The polynucleotide 5′-phosphate may comprise one or more of RNA species selected from small RNAs; small nuclear RNAs (snRNAs); small nucleolar RNAs (snoRNAs); miRNAs; Piwi-interacting RNAs (piRNAs); lncRNAs; tRNAs; ribosomal RNAs (rRNAs); mRNAs; non-coding RNAs (ncRNAs); intergenic RNAs; silencing RNAs (siRNAs); small regulatory RNAs (srRNAs); or any combinations thereof. The polynucleotide 5′-phosphate may comprise samples of fragmented and/or degraded RNAs, particularly fragmented and/or degraded mRNAs and long noncoding RNAs. The polynucleotide 5′-phosphate may comprise fragmented and/or degraded DNA, such as ancient DNA, environmental DNA, forensic DNA, circulating DNA (e.g., exosomes), denatured DNA, and viral DNA. Several of these DNAs and RNAs may be used as biomarkers and in medical diagnosis applications. These DNAs and RNAs may be obtained from a cell or tissue extract; or from formalin-fixed, paraffin-embedded tissue (FFPE); or from a body fluid, such as saliva, blood, menstrual blood, cervicovaginal fluid, and semen.

The polynucleotide population may include polynucleotide 5′-phosphates having one or more polynucleotides with different 5′ termini. The polynucleotide 5′-phosphate will be selectively capped by the reaction with a nucleoside 5′-phosphoroimidazolide, while the other polynucleotides in the population will remain unreactive. In other embodiments, it may be desirable to cap polynucleotides with no terminal phosphate at the 5′ end; in such cases, these polynucleotides may be pre-treated with a polynucleotide kinase to install a 5′ terminal phosphate to those polynucleotides lacking a 5′ phosphate. In other embodiments, it may be desirable to repair the 3′ end of a polynucleotide, i.e., to remove a terminal 3′ phosphate or a 2′,3′-cyclic phosphate, to avoid unwanted formation of 3′ to 5′ polyphosphate linkage by reaction with a nucleoside 5′-phosphoroimidazolide; in such cases, these polynucleotides may be pre-treated with a polynucleotide kinase (e.g., T4 PNK), or a related enzyme (e.g., a phosphatase) that is able to dephosphorylated a 3′ terminal nucleotide or 2′,3′-cyclic phosphate to create a 3′ OH terminus (Zhelkovsky, (2014) J Biol Chem 289: 33608-16).

In some embodiments, it may be desirable to replace in a polynucleotide an existing 5′ cap structure such as the N7-methylguanosine cap in eukaryotic mRNA; or a trimethylguanosine cap in small nuclear RNAs (snRNAs); or a γ-methyl phosphate cap in snRNAs, such U6 and 7SK; or cap-like structures such as nicotinamide adenine dinucleotide (NAD⁺), 3′-desphospho-coenzyme A (dpCoA), and other moieties attached to the 5′ end of RNA by an oligophosphate bridge [Warminski, et al. (2017) Top Curr Chem 375: 16]. In such cases, the existing cap structure is replaced by a process of decapping the 5′ end so that the polynucleotides in the population have a terminal phosphate at the 5′ end (e.g., a 5′-monophosphate, a 5′-diphosphate, and/or a 5′-triphosphate) and this terminal 5′-phosphate then re-capped by chemical capping. (also see US 2018/0195061). The decapping reaction may be mediated by an enzyme or by chemical hydrolysis (appropriate acidic or basic conditions may be selected). When the decapping reaction is carried out enzymatically, the enzyme may be selected from a deadenylase, an apyrase, a 5′RppH, an Nudix phosphohydrolase, a tobacco acid polyphosphatase, a member of the HIT superfamily of pyrophosphatases, a DcpS, a Dcp1-Dcp2 complex, a NudC, an APTX, a member of the DXO family proteins, a APAH-like phosphatases, a Cap-Clip reagent (CELLSCRIPT, Madison, Wis.), or a combination thereof. Examples and uses of decapping enzymes are reviewed in Kramer, et al. (2019) Wiley Interdiscip Rev RNA, 10: e1511.

The chemical capping method described above can be incorporated into a variety of cDNA synthesis methods. For example, the RNA may be chemically capped, copied into cDNA, and then sequenced. In some embodiments, the cDNA synthesis may be performed in the presence of a TSO, thereby producing cDNAs that contain the complement of the TSO at the 3′ end. Accordingly, the RNA may be cellular RNA that has been extracted obtained from cells, e.g., mammalian cells directly, for example. Such a sample may contain a population of different naturally occurring RNA molecules, or fragments thereof, in which case it may contain more than 1,000, more than 10,000, more than 50,000, or more than 100,000 up to 1M or more different species of RNA, i.e., RNA molecules of different sequence. The RNA may contain mRNA molecules, which are typically at least 100 nt in length (e.g., 200 nt to 10 kb in length) and have a median length in the range of 500-5,000 nt. An RNA sample may additionally contain a variety of small non-coding regulatory RNAs that may be generically referred herein to as “small RNAs”, e.g., short interfering RNAs, microRNAs, tiny non-coding RNAs, piwi-interacting small RNAs (piRNAs), snoRNAs and small modulatory RNAs Small RNAs are typically below 100 nt in length and have a median length in the range of 18 nt to 40 nt. An RNA sample may additionally contain rRNA molecules, tRNA molecules, pre-miRNA molecules, snRNAs and long non-coding RNA (lncRNA) molecules such as large intergenic RNA (lincRNA) molecules.

The method described herein can be employed to analyze polynucleotide samples from virtually any organism and/or sample-type, including, but not limited to, plants, animals (e.g., reptiles, mammals, insects, worms, fish, etc.), viruses, tissue samples, bodily fluids, cadaveric tissue, archaeological/ancient samples, etc. In certain embodiments, the polynucleotide sample used in the method may be derived from a mammal, where in certain embodiments the mammal is a human. In exemplary embodiments, the polynucleotide sample may contain RNA and/or DNA from a mammalian cell, such as, a human, mouse, rat, or monkey cell. The sample may be made from a biopsy of tissue or a liquid biopsy or from cultured cells, fixed cells or cells of a clinical sample, e.g., a tissue biopsy, scrape or lavage or cells of a forensic sample (i.e., cells of a sample collected at a crime scene). The sample may include or target exosomes. The sample may include a blood sample from a pregnant woman to analyze fetal polynucleotides. In particular embodiments, the RNA and/or DNA sample may be obtained from a biological sample such as cells, tissues, bodily fluids, and stool. Bodily fluids of interest include but are not limited to, blood, serum, plasma, saliva, mucous, phlegm, cerebral spinal fluid, pleural fluid, tears, lactal duct fluid, lymph, sputum, cerebrospinal fluid, synovial fluid, urine, amniotic fluid, and semen. In particular embodiments, a sample may be obtained from a subject, e.g., a human. Template switching using chemical capping as described herein may be used in diagnostic tests for cancer, genetic conditions, chronic disorders, autoimmune disorders, infectious agents (e.g., E. coli, influenza, and SARS-CoV-2 among others), environmental contamination by biological material, microbiome or other bacterial populations, the effects of therapeutic agents or drugs on gene expression or the effect of any external stimuli or developmental effect on gene expression in an organism etc. Amplification steps used in the methods and uses described herein may include any amplification method known in the art such as temperature cycling methods such as PCR, isothermal amplification such as LAMP, HDA, or ligase dependent amplification methods.

Any of the reagents may be immobilized to facilitate the workflow. For example, template switching reverse transcription and/or adaptor ligation steps may be carried out by immobilized enzymes, such as immobilized RTs and DNA polymerases, and immobilized ligases and poly(A) polymerases, respectively. The enzymes may be immobilized in a solid surface by fusion to a protein tag, such as SNAP-tag or CLIP-tag, followed by reaction with an appropriately functionalized solid surface, such as magnetic bead modified with a SNAP-tag reactive O6-benzylguanine (BG) functional group or CLIP-tag reactive O2-benzylcytosine (BC) functional group (see immobilization of enzymes on magnetic microbeads using SNAP-tag, see for example Li, et al (2018) Bioconjugate Chemistry, 29: 2316-2324).

The chemical coupling of a cap structure to a polynucleotide through a 5′ to 5′ polyphosphate linkage enhances the incorporation of 3′ sequencing adaptors to cDNA by TS. Sequencing results show that the embodiments provide sensitivity and specificity (see for example, FIG. 8A through FIG. 13C) for detecting and quantifying RNA or DNA polynucleotides. The efficiency of TS increased by more than 1.5-fold or between 2-fold and 5-fold for capped RNAs compared to uncapped RNAs (see for example, FIG. 5A-5B). TS efficiency of chemically capped polynucleotides exceeded 30% efficiency. Moreover, as a measure of reduced bias, the sequences of at least 40% of the capped molecules and as much as 75% or more of the capped molecules fell within 2-fold expected variation regardless of the identity of the two terminal nucleotides behind the cap (see for example FIG. 9 and Example 10).

In some cases, the chemically 5′-capped polynucleotide may be used for direct sequencing without cDNA synthesis. Bypassing the need for cDNA synthesis is desirable so as to avoid amplification and thus eliminate PCR bias or RT bias. Additionally, typical direct RNA or DNA sequencing is compatible with very long reads, which are particularly useful, for instance, in the study of transcription initiation sites (TSS) and splice variants.

Sequencing of the chemically 5′-capped RNA or DNA may be performed through a nanopore device as an example of direct sequencing. In some cases, the 5′-cap structure may further comprise an adaptor sequence (e.g., an oligonucleotide attached at 2′ or 3′ position of the nucleoside, or at any of the positions of the nucleobase), where the attached adaptor oligonucleotide facilitates reading the sequences at and around the 5′ end of the target RNA. In other cases, the 5′-cap structure may further comprise a reactive group (e.g., an alkyne or azide) that enables attachment of an adaptor oligonucleotide to the target RNA after the capping reaction.

Any sequencing platform known in the art may be used without limitation. In addition to Oxford Nanopore sequencing, other examples include Illumina sequencing, sequencing using a Pacific Biosciences sequencer or a Beijing Genome Institute sequencer.

The chemically 5′-capped polynucleotide may be formed by coupling between an activated nucleoside 5′-(mono, di or tri)phosphate with a polynucleotide 5′-(mono, di or tri)phosphate, where the polynucleotide 5′-(mon, di or tri)phosphate is (i) naturally present in eukaryotic RNA, prokaryotic RNA, or mixture thereof; (ii) obtained from decapping of any naturally capped mRNA molecules in the sample to produce the 5′-diphosphorylated or 5′-monophsophorylated RNA molecules; or (iii) obtained from 5′ phosphorylation of any 5′-unphosphorylated RNA molecules in the sample, whether naturally occurring or produced from fragmentation or degradation of the sample.

Where the activated nucleoside 5′-phosphate further comprises a suitable affinity tag (e.g., biotin or desthiobiotin), this enables enrichment of chemically 5′-capped polynucleotides in a population. In certain embodiments, the affinity group may combine with an adaptor oligonucleotide in the same molecule so as to allow enrichment and sequencing of the chemically 5′-capped target polynucleotide.

In some embodiments, the chemically 5′-capped polynucleotide can be subsequently manipulated. For example, the chemically 5′-capped polynucleotide can be isolated (captured, purified, enriched) by, for example affinity binding to a suitable matrix. Any suitable matrix can be used, such as and without limitation, a solid, semi-solid, or porous matrix. The matrix can be in any suitable form such as beads including magnetic beads, column, plate, or microfluidic device. Such matrices can be treated, adsorbed, affinity coated, with a binding reagent, ligand or labeling partner specific for binding the label on the mononucleotide. The matrix may be made of any suitable materials, including metal, polystyrene, glass, paper, protein or other biological or chemical reagent such as a polymer. Once bound to the matrix, the bound chemically 5′-capped polynucleotide can be washed, eluted or otherwise isolated and optionally purified from the mixture for subsequent analysis as desired. Enrichment by immobilization on a matrix can be achieved at temperatures in the range of 25° C. to 80° C., for example, 25° C. to 75° C. or 30° C. to 60° C.

In some embodiments, the 5′-capped polynucleotide according to the methods described herein may be fragmented before or after capping. Such fragmenting reduces the sizes of the polynucleotide to any desired length. For example, the polynucleotide fragments can be around 10-10000 nucleotides in length, or ranges in between, e.g., 100-1000 nucleotides, 10-500 nucleotides, 3000-5000 nucleotides, or about 50, 100, 200, 250 nucleotides. Fragmenting can be achieved using standard techniques, including mechanical shearing, chemical, enzymatic digestion and sonication.

A composition or kit may contain at least: a) an activated guanosine 5′-phosphate reagent, as described above; and b) a capping reaction buffer in the range of pH 5-pH 6.5, as described above.

The composition or kit components may be added to a substrate comprising a naturally occurring polynucleotide in a population of polynucleotides or a polynucleotide that has been decapped or 5′-phosphorylated, as described above to form a second composition.

The composition or kit may include a plurality of modules, each module being in one or more tubes, wherein a first module is a capping module comprising reagents for chemically capping a population of polynucleotide, and a second module comprising a cDNA synthesis and amplification module, wherein the second module optionally includes a TSO and/or an 3′ adaptor oligonucleotide (such as 3′ splint adaptor or a 3′ random adaptor). A composition or kit may further include a polynucleotide kinase and/or a poly(A) polymerase.

Representative examples of workflows for the synthesis of a cDNA first strand from target DNA or RNAs in a population of polynucleotides, such as occur in a cell lysate, are shown in FIG. 4A-4D. In each workflow a different strategy was used to incorporate an adaptor sequence to the 5′ end of the cDNA. In all workflows, the addition of an adaptor sequence at the 3′ end of the cDNA is the same and involve a step of chemical capping the 5′ end of the target DNA or RNA prior to TS by a RT. The product of TS is a cDNA first strand comprising 5′- and 3′-adaptor sequences. After synthesis of a cDNA second strand, a PCR amplification step may be followed to prepare the cDNA for deep-sequencing platforms.

In some instances, it may be desirable to omit the PCR amplification to avoid introducing artifacts into sequencing libraries. This is particularly important when sequencing genomes or genomic regions with highly biased base composition such as the genomes of Plasmodium falciparum with a high adenine-thymine (AT) content and Mycobacterium tuberculosis with high guanine-cytosine (GC) content.

Prior to the synthesis of the cDNA by an RT, an adaptor of some sort may be added to the 3′ end of the polynucleotide to provide a priming site for the RT. There are several strategies to incorporate an adaptor sequence that also provides a priming site for the RT as outlined in FIG. 4A-4D (see for example Zhuang, et al. (2012) Nucleic Acids Res. 2012, 40: e54).

FIG. 4A shows a workflow for adding an adaptor sequence (DNA Primer) that has a portion that is either a randomized sequence or matches a segment of the 3′ end of the target DNA or RNA to initiate the reverse transcription reaction. This adaptor sequence also comprises a portion that has a defined sequence for PCR amplification or direct sequence.

FIG. 4B shows a workflow for adding a polyA tail (e.g., SEQ ID NO:1) by means of a Poly(A) Polymerase to 3′ end of the 5′ chemically capped RNA, and then synthesizing the cDNA using a DNA primer that contains a poly(dT) segment to initiate the reverse transcription reaction. A polyA tail may be naturally present on a polynucleotide such as eukaryotic mRNA. Alternatively, a nucleotide homopolymer sequence can be added using a poly(A) polymerase or terminal deoxyribonucleotidyl transferase. For instance, a polyG, polyC, or polyU tail may be added to the DNA or RNA template through the use of Poly(U) Polymerase. In such cases, a polydC, polydG, or polydA primer, respectively, can be used for RT priming.

FIG. 4C shows a workflow in which a 3′-Adaptor is ligated to the 3′ end of the target DNA or RNA for the synthesis of a cDNA first strand that involves adding a RT primer that hybridizes to the ligated sequence. This 3′ adaptor may comprise one or more randomized nucleotides up to 10 randomized nucleotides. A particularly preferred 3′ adaptor comprises 4 to 6 randomized nucleotides. This 3′ adaptor may also a comprise a pool of two or more 3′ adaptors comprising randomized nucleotides. A particularly preferred pool of 3′ adaptor comprises between two and twenty 3′ adaptors, each adaptor sequence containing between 4 and 6 randomized nucleotides. FIG. 4D shows a workflow for ligating a 3′ adaptor sequence to the polynucleotide using a splinted adaptor (see for example, U.S. Provisional Application No. 62/839,191). An example of a 3′-Splint Adaptor for use herein includes a DNA having a top strand with a 5′-pre-adenylated end (5′-App-) and a 3′ blocked end (e.g., 3′ amino-linker, a 3′ inverted nucleotide, or a 3′-dideoxynucleotide), and a bottom strand with a 3′ blocked end (e.g., 3′ amino-linker, a 3′ inverted nucleotide, or a 3′-dideoxynucleotide) followed by 4 to 8 randomized nucleotides, preferentially 6 randomized nucleotides, which are then followed by a deoxyribouridine nucleotide and a pre-defined sequence that hybridize with the top strand. The randomized nucleotides may then be removed by cleavage at the deoxyribouridine site through USER® enzyme (New England Biolabs, Ipswich, Mass.) cutting to create a primer for reverse transcription. The top strand of 3′-Splint Adaptor may comprise a 5′-pre-adenylated end (5′-App-) as described above or, alternatively, a 5′-monophosphate end. A 3′-Splint Adaptor comprising a top strand 5′-monophosphate end is particularly useful for DNA templates, when combined with DNA ligases (e.g., T4 DNA ligase). FIG. 4D shows a workflow in which a 3′-Splint Adaptor that is a double-stranded nucleic acid with a 3′-extension segment containing randomized nucleotides is ligated to the 3′ end of the target template. In other embodiments, the strategy of ligating a 3′ adaptor sequence to the RNA or DNA template employs a splinted adaptor sequence, where the adaptor consists of a single oligonucleotide folded in a hairpin-shaped structure.

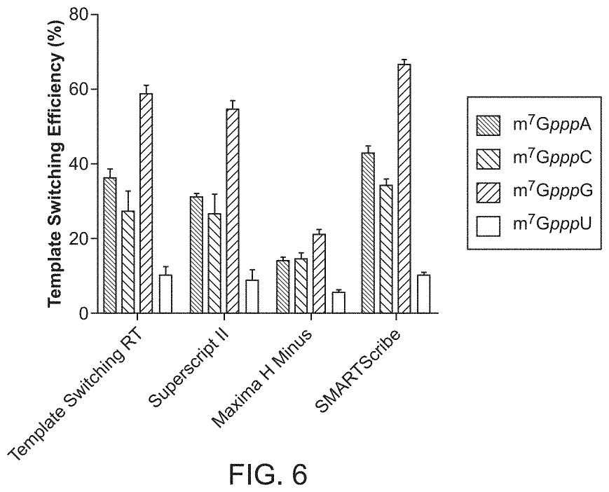

The RT may be selected from one of the many families of RTs, including retrovirus RTs such as human immunodeficiency virus (HIV) RT, Avian Myeloblastosis Virus (AMV) RT, Moloney Murine Leukemia Virus (MMLV) RT, and Jaagsiekte sheep retrovirus (JSRV) RT ([Yu, et al. (1992) J Biol Chem 1992, 267: 10888-96; Gerard, et al. (1997) Mol Biotechnol 1997, 8: 61-77]; recombinant endogenous retrovirus RTs, such as the RT derived from the recombination between the N-tropic provirus located at the Emv-1 genetic locus and a B-tropic endogenous murine leukemia virus in Neuro-2a cell line (Pothlichet, et al. (2006) Int. J. Cancer 119: 815-822); Telomerase RTs; Retrotransposon RTs (Belfort, et al. (2011) Proc Natl Acad Sci USA 108: 20304-10); Non-LTR retro element RTs; and Group II Intron RTs (Nottingham, et al. (2016) RNA 2016, 22: 597-613). Additional examples of retroviruses that can serve as sources of RTs, include bovine immunodeficiency virus (BIV), caprine encephalitis-arthritis virus (CAEV), equine infectious anemia virus (EIAV), feline immunodeficiency virus (FIV), goat leukoencephalitis virus (GLV), Jembrana virus (JDV), maedi/visna virus (MVV), and progressive pneumonia virus (PPV). Also encompassed are viruses such as hepatitis B virus (HBV) that although not technically classified as retroviruses, nonetheless utilize a RT. Several of these RTs are commercially available, including Transcriptor RT (Roche Diagnostics, Indianapolis, Ind.), AMV and AMV XL RTs, Protoscript® II (New England Biolabs, Ipswich, Mass.), Superscript® RTs (ThermoFisher Scientific, Waltham, Mass.), SMARTScribe™ RT (Takara Bio, Mountain View, Calif.), Expand™ RT (Sigma Aldrich, St. Louis, Mo.), TGIRT™-III Enzyme (InGex, St. Louis Mo.), among others. Particularly useful are RNase H-deficient and/or thermostable RT enzymes that are able to read through modified nucleotides and secondary structures, such as stem-loops formed by small RNAs, that may impede uniform reverse transcription of all members of the polynucleotide pool. Most preferable are RTs that have the ability to perform inter-strand TS efficiently. Examples of RTs are shown in FIG. 6.

The reverse transcription may also be performed by a DNA Polymerase that possesses reverse transcription activity. Examples of such enzymes include but are not limited to members of family A DNA polymerases such as: Bst, Taq, Tth, Klenow, KOD, Sco, and Sli; topoisomerases such as Sco TopA and Sli TopA; Phi29 DNA polymerase; and combination thereof. The reverse transcription activity in DNA polymerases may further be enhanced by replacing magnesium ions in the reaction buffer with manganese ions. Examples of DNA polymerases displaying reverse transcription activity have been reported by Myers, et al. (1991) Biochemistry 30: 7661-6; Grabko, et al. (1996) FEBS Lett 387: 189-92; and Bao, et al. (2004) Proc Natl Acad Sci USA 101: 14361-6. Most preferable are DNA polymerases that have the ability to perform inter-strand TS efficiently.

The reverse transcription may also be performed by a combination of a RT with TS activity and a high-fidelity DNA polymerase to enable the synthesis of a high-fidelity cDNA product from the polynucleotide template. This approach is suitable for determining single nucleotide polymorphisms (SNP) in the polynucleotide template.

A 3′ DNA adaptor sequence complementary to the TSO is incorporated into the first strand cDNA during TS reverse transcription by the RT. In some embodiments, the TSO may be DNA, or RNA, or a chimeric polynucleotide (chimera) consisting of RNA and DNA bases. Preferably, the TSO is a hybrid oligonucleotide where nucleotides at the 5′-end are DNA and last 3 nucleotides at the 3′ end are RNA. In other embodiments, the RNA nucleotides at the 3′ end of the TSO may be further modified to increase the strength of hybridization of between TSO and cDNA strand. Examples of such modifications include but are not limited to 2′-fluoro-2′-deoxy nucleotides and 2′-methoxy nucleotides. Examples of TSO 3′ends include rGrGrG; rUrUrG; rGrUrG; ^{2′F}rG^{2′F}rG^{2′F}rG; rCrCrC; rUrUrU; rIrIrI; ^{2′OMe}rG^{2′OMe}rG^{2′OMe}rG; ^{2′F}rG^{2′F}rU^{2′F}rG; ^{2′F}rG^{2′F}rG^{2′F}rA; ^{2′F}rG^{2′F}rG^{2′F}rC; and ^{2′F}rG^{2′F}rG^{2′F}rU; where I is inosine, ^{2′F}N is a 2′-fluoro-2′-deoxy nucleotide, and ^{2′OMe}N is 2′-methoxy nucleotide. Further examples of TSO 3′ends include dGdGdG^{CLAMP}; dGdG-methylene blue; and dGdTdG-azide; where dG^{CLAMP} is a tricyclic aminoethyl-phenoxazine 2′-deoxycytidine analogue synthesized using the AP-dC-CE Phosphoramidite (Glen Research, Sterling, Va.). More examples of base, sugar and/or phosphate modifications, including the use of carbocyclic and acyclic analogues have been listed earlier in the invention and also apply here for 3′ end TSO modifications.

In further embodiments, TSOs may also include modifications at the 5′ end such as: a 5′-rU, a 5′-Biotin, a 5′-Biotin-d^{iso}G, a 5′-N₃, or a 5′-dA-(Int Spacer 9), wherein Int Spacer 9 is a triethylene glycol spacer from Integrated DNA Technologies (Coralville, Iowa), and i^{so}G represents an isoguanosine. Nucleotide modifications at the TSO 5′ end are designed to decrease TSO concatemerization on the cDNA strand.

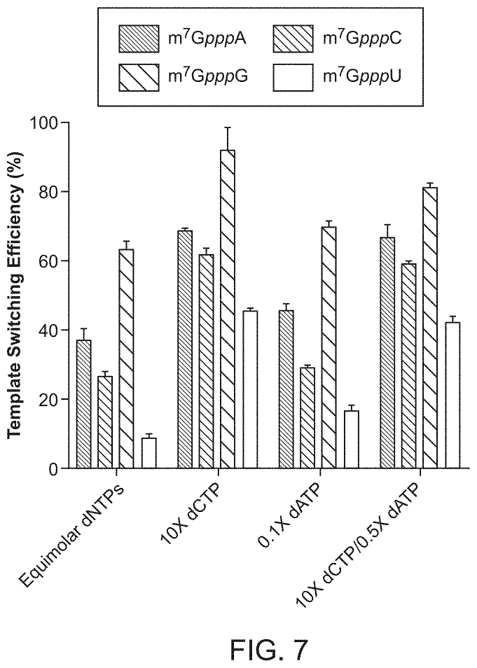

The cDNA synthesis by template switching reverse transcription may be performed in the presence of equimolar or nonequimolar dNTP mixtures as shown in FIG. 7. In some embodiments, the dNTP mixture may comprise of an excess of each of dATP, dCTP, dGTP, or dTTP, where the relative composition of each dNTP may vary from 1.1 to 100-fold, preferably 10-fold (10×). The most preferred condition is a 10-fold excess of dCTP (10×dCTP) relative to other dNTPs. In some embodiments, the dNTP mixture may also comprise of an undersupply of each of dATP, dCTP, dGTP, or dTTP, where the relative composition of each dNTP may vary from 0.1 to 0.001-fold, preferably 0.1-fold (0.1× or 1/10). The most preferred condition is a 0.1-fold undersupply of dATP (0.1×dATP) relative to other dNTPs. Any combinations of excess and/or undersupply of dNTPs may be employed in any of the embodiments.

The method for synthesis of a cDNA from a polynucleotide (e.g., an DNA or RNA nucleic acid) by template switching reverse transcription comprising the steps of: (a) selecting a 5′ cap precursor comprising a nucleotide, or modifications thereof, linked to a phosphate, and optionally a leaving group such as an imidazole; (b) chemically linking a 5′ cap precursor to the 5′ end of the target nucleic acid through a 5′ to 5′ polyphosphate linkage to form a 5′ capped nucleic acid; (c) optionally treating the 5′ capped nucleic acid with a 5′→3′ exonuclease (e.g., XRN-1, Terminator, Rexo5, Exo T, TREX1, and related exonucleases that specifically degrade polynucleotide 5′-monophosphates) to degrade any remaining uncapped sequences in the polynucleotide population; (d) ligating a 3′ adaptor to the 5′ capped nucleic acid wherein the adaptor optionally contains a cDNA priming site; (e) contacting the 5′ capped nucleic acid of (b) or (c) or (d) with a TSO and a RT; and (e) synthesizing the DNA complementary to the target nucleic acid. The method may further comprise size-selecting or enriching the target nucleic acid before the step of (b).

The product of template switching reverse transcription is a cDNA first strand comprising 5′- and 3′-adaptor sequences. After synthesis of a cDNA second strand, a PCR amplification step may be followed to prepare the cDNA for deep-sequencing platforms. The amplicons may be subjected to further library preparation methods according to workflow and instructions provided by specific sequencing platforms, including but not limited to Illumina (San Diego, Calif.) (e.g., iSeq®, MiniSeq®, MiSeq®, HiSeq®, NovaSeq®, and NextSeq®), ThermoFisher Scientific (Waltham, Mass.) (e.g., Proton™ I and PGM), Pacific Biosciences (Menlo Park, Calif.) (e.g., PacBio Sequel® and RSII), Roche 454, SOLiD, and Oxford Nanopore Technologies (Oxford, UK) (e.g, MinION®, GridION®, PromethION®, and Flongle®). For example, the cDNA may be amplified and then sequenced on a substrate using, e.g., Illumina's reversible dye terminator method, the cDNA may be sequenced using a single molecule sequencing method.

### Kits

Also provided by this disclosure are kits for practicing the subject method, as described above. In some embodiments, the kit may additionally contain any one or more of the components listed above. For example, a kit may include an activated guanosine 5′ mono- or poly-phosphate and capping reaction buffer that has a pH in in the range of pH 5-pH 6.5; a nucleoside 5′-phosphoroimidazolide, a capping buffer pH 5-pH 6.5, and a naturally occurring population of polynucleotides; nucleoside 5′-phosphoroimidazolide, a capping buffer pH 5-pH 6.5, a RT and optionally a TSO; and or a plurality of modules, each module being in one or more containers, wherein a first module is a capping module comprising reagents for chemically capping a population of polynucleotide, and a second module comprising a cDNA synthesis and amplification module, wherein the second module optionally includes (i) a TSO and/or a 3′ splint adaptor. A kit may also contain one or more primers, e.g., a primer for making first strand cDNA, RT etc. The various components of the kit may be present in separate containers or certain compatible components may be precombined into a single container, as desired. In addition to above-mentioned components, the subject kits may further include instructions for using the components of the kit to practice the subject methods, i.e., to instructions for sample analysis.

All publications, patents, and patent applications mentioned in this specification are herein incorporated by reference to the same extent as if each individual publication, patent, or patent application was specifically and individually indicated to be incorporated by reference.

### EXAMPLES

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. To exemplify the claimed invention, figures have been provided and described in some detail above. The results they demonstrate may be achieved using the methods described below.

### Example 1: Preparation of RNA Templates, Template Switching Oligonucleotides (TSOs) and DNA Primers

5′-hydroxyl (5′-OH) and 5′-monophosphate (5′-p) RNAs, TSOs, RT DNA primers and adaptors were synthesized by Integrated DNA Technologies (Coralville, Iowa). 5′-triphosphate RNAs were synthesized according to Goldeck, et al., Angew. Chem. Int. Ed. 53, 4694-4698 (2014). The sequence of RT primers, TSOs, and adaptors are described in Table 1.

TABLE 1
RT Primer, TSO, and Adaptor Sequences (to differentiate
DNA from RNA nucleotides in this table, RNA nucleotides
are preceding by “r”, DNA nucleotides are preceding by
“d”; “dd” represents a dideoxynucleotide).

| | Sequence |
|---|---|
| RT Primer | |
| 5′-FAM | 5′-FAM-d(TTGAGCGTACTCGACGAAGT)-3′ (SEQ ID NO: 2) |
| DNA primer | |
| i7 Primer | 5′-d(GACTGGAGTTCAGACGTGTGCTCTTCCGATCTNNNNNNTTGAGCGTACT |
| | CGACGAAG)-3′(SEQ ID NO: 3) |
| Poly(dT) | 5′-d(AGACGTGTGCTCTTCCGATCTTTTTTTTTTTVN)-3′ |
| primer | (SEQ ID NO: 4) |
| SR RT | 5′-d(AGACGTGTGCTCTTCCGATCT)-3′ (SEQ ID NO: 5) |
| primer | |
| TSO | |
| rGrGrG-3′ | 5′-d(GCTAATCATTGCAAGCAGTGGTATCAACGCAGAGTACAT)rGrGrG-3′ |
| TSO | (SEQ ID NO: 6) |
| rUrUrG-3′ | 5′-d(GCTAATCATTGCAAGCAGTGGTATCAACGCAGAGTACAT)rUrUrG-3′ |
| TSO | (SEQ ID NO: 7) |
| rGrUrG-3′ | 5′-d(GCTAATCATTGCAAGCAGTGGTATCAACGCAGAGTACAT)rGrUrG-3′ |
| TSO | (SEQ ID NO: 8) |
| ^{2′F}rG^{2′F}rG | 5′-d(GCTAATCATTGCAAGCAGTGGTATCAACGCAGAGTACAT)^{2′F}rG |
| ^{2′F}rG-3′ TSO | ^{2′F}rG^{2′F}rG-3′ (SEQ ID NO: 9) |
| i5-rGrGrG-3′ | 5′-d(ACTAATCATTGCTACACTCTTTCCCTACACGACGCTCTTCCGATCT)rG |
| TSO | rGrG-3′ (SEQ ID NO: 10) |
| Adaptor | |
| 3′-SR | 5′-rApp-d(AGATCGGAAGAGCACACGTCT)-NH2-3′ (SEQ ID |
| Adaptor | NO: 11) |
| 3′-Random | 5′-rApp-d(TCGTATGNNNNNNAGATCGGAAGAGCACACGTCT)-NH₂-3′ |
| Adaptor | (SEQ ID NO: 12) |
| (equal | 5′-rApp-d(AGCATACNNNNNNAGATCGGAAGAGCACACGTCT)-NH₂-3′ |
| mixture of | (SEQ ID NO: 13) |
| these four | 5′-rApp-d(CTACGCANNNNNNAGATCGGAAGAGCACACGTCT)-NH₂-3′ |
| sequences) | (SEQ ID NO: 14) |
| | 5′-rApp-d(GATGCGTNNNNNNAGATCGGAAGAGCACACGTCT)-NH₂-3′ |
| | (SEQ ID NO: 15) |
| 3′-Splint | 5′-rApp-d(AGATCGGAAGAGCACACGTCT)-ddC-3′ |
| Adaptor (for | (SEQ ID NO: 16) |
| RNA | 3′-NH₂-d(NNNNNNUCTAGCCTTCTCGTGTGCAGA)-5′ |
| templates) | (SEQ ID NO: 17) |
| 3′-Splint | 5′-p-d(AGATCGGAAGAGCACACGTCT)-ddC-3′ (SEQ ID NO: 18) |
| Adaptor (for | |
| DNA | 3′-NH₂-d(NNNNNNUCTAGCCTTCTCGTGTGCAGA)-5′ (SEQ ID |
| templates) | NO: 19) |

### Example 2: Synthesis of Nucleoside 5′-Phosphoroimidazolides

A nucleoside 5′-phosphate (0.9 mmol), imidazole (9 mmol), 2′,2′-dithiodipyridine (3.5 mmol), and triphenylphosphine (3.5 mmol) were combined. Anhydrous DMF (7.0 mL) and triethylamine (3.5 mmol) was added, and mixture stirred at room temperature overnight. Lithium perchlorate (7 g) in acetone (70 mL) was added. The suspension was cooled to 4° C. Following centrifugation, the supernatant was removed, and the precipitate was washed with cold acetone and then dried under vacuum. Table 2 lists ribonucleoside 5′-phosphates converted to corresponding imidazolides and provides yield and purity after isolation for each phosphoroimidazolide. Abbreviations are as follows: AMP, adenosine 5′-monophosphate; ADP, adenosine 5′-diphosphate; ATP, adenosine 5′-triphosphate; CMP, cytidine 5′-monophosphate; CDP, cytidine 5′-diphosphate; CTP, cytidine 5′-triphosphate; GMP, guanosine 5′-monophosphate; GDP, guanosine 5′-diphosphate; GTP, guanosine 5′-triphosphate; UMP, uridine 5′-monophosphate; UDP, uridine 5′-diphosphate; UTP, uridine 5′-triphosphate; IDP, inosine 5′-diphosphate.

TABLE 2
Conversion of ribonucleoside 5′-phosphates to 5′-phosphoroimidazolides

| Nucleoside 5′- | | Imidazolide | | | |
|---|---|---|---|---|---|
| phosphate | Activation | product | Mass (mg) | Yield (%) | Purity (%) |
| AMP | imidazole | imAMP | 76.4 | 46.9 | 97.0 |
| CMP | imidazole | imCMP | 107.7 | 61.1 | 98.6 |
| GMP | imidazole | imGMP | 3602.7 | 91.0 | 74.0 |
| ADP | imidazole | imADP | 138.1 | 55.0 | 97.0 |
| CDP | imidazole | imCDP | 92.0 | 56.1 | 94.1 |
| GDP | imidazole | imGDP | 294.4 | 99.1 | 91.5 |
| UDP | imidazole | imUDP | 164.5 | 61.0 | 96.7 |
| ATP | imidazole | imATP | 120.2 | 60.7 | 93.9 |
| CTP | imidazole | imCTP | 102.1 | 63.3 | 95.7 |
| GTP | imidazole | imGTP | 110.1 | 62.1 | 96.4 |
| UTP | imidazole | imUTP | 121.7 | 60.8 | 97.6 |
| IDP | imidazole | imIDP | 106.6 | 62.3 | 96.0 |
| GDP | 2-methylimidazole | MeimGDP | 112.2 | 54.4 | 93.9 |
| GDP | benzimidazole | BnimGDP | 102.9 | 53.1 | 95.0 |

### Example 3: Chemical Capping of RNAs

Sixteen different 5′-monophosphate 25mer RNAs (5′-p-NNAGAACUUCGUCGAGUACGCUCAA-3′ (SEQ ID NO:20), wherein N is G, C, A, or U) were made by standard solid-phase synthesis. Table 3 lists the sequences of each individual synthetic oligonucleotide 5′-monophosphate RNA. Chemical capping of each individual 5′-monophosphate RNA (5 nmol) was carried out in a 250 tit reaction mixture having 100 μM 5′-monophosphate RNA in a chemical capping buffer pH 6.0 containing an organic cosolvent, to which was added a 100 mM solution of an imidazolide-NMP, -NDP, or -NTP. The capping reaction was incubated either at 50° C. for 5 hours (imNMPs), 37° C. for 4 hours (imNDPs), or room temperature for 4 hours (imNTPs). The phosphoroimidazolides used in this coupling reaction are listed in Table 2. After this time, the unreacted phosphoroimidazolide was removed from the reaction along with salts and organic solvent using a Sep-Pak C18 Cartridge (Waters, Milford, Mass.). The capped oligonucleotide was eluted from the column using 1:1 TEAB:Acetonitrile (2 mL) and concentrated using a SpeedVac® (ThermoFisher Scientific, Waltham, Mass.). Each 5′-capped RNA was purified by polyacrylamide gel electrophoresis and had its identity confirmed by mass spectrometry (Oligo HTCS, Novatia, Newtown, Pa.).

TABLE 3
Sequences of synthetic oligonucleotide
5′-monophosphate RNAs

| Oligo- | |
|---|---|
| nucleotide | |
| ID | Sequence |
| AA | 5′-p-AAAGAACUUCGUCGAGUACGCUCAA-3′ |
| | (SEQ ID NO: 21) |
| AC | 5′-p-ACAGAACUUCGUCGAGUACGCUCAA-3′ |
| | (SEQ ID NO: 22) |
| AG | 5′-p-AGAGAACUUCGUCGAGUACGCUCAA-3′ |
| | (SEQ ID NO: 23) |
| AU | 5′-p-AUAGAACUUCGUCGAGUACGCUCAA-3′ |
| | (SEQ ID NO: 24) |
| CA | 5′-p-CAAGAACUUCGUCGAGUACGCUCAA-3′ |
| | (SEQ ID NO: 28) |
| CC | 5′-p-CCAGAACUUCGUCGAGUACGCUCAA-3′ |
| | (SEQ ID NO: 29) |
| CG | 5′-p-CGAGAACUUCGUCGAGUACGCUCAA-3′ |
| | (SEQ ID NO: 30) |
| CU | 5′-p-CUAGAACUUCGUCGAGUACGCUCAA-3′ |
| | (SEQ ID NO: 31) |
| GA | 5′-p-GAAGAACUUCGUCGAGUACGCUCAA-3′ |
| | (SEQ ID NO: 32) |
| GC | 5′-p-GCAGAACUUCGUCGAGUACGCUCAA-3′ |
| | (SEQ ID NO: 33) |
| GG | 5′-p-GGAGAACUUCGUCGAGUACGCUCAA-3′ |
| | (SEQ ID NO: 34) |
| GU | 5′-p-GUAGAACUUCGUCGAGUACGCUCAA-3′ |
| | (SEQ ID NO: 35) |
| UA | 5′-p-UAAGAACUUCGUCGAGUACGCUCAA-3′ |
| | (SEQ ID NO: 36) |
| UC | 5′-p-UCAGAACUUCGUCGAGUACGCUCAA-3′ |
| | (SEQ ID NO: 37) |
| UG | 5′-p-UGAGAACUUCGUCGAGUACGCUCAA-3′ |
| | (SEQ ID NO: 38) |
| UU | 5′-p-UUAGAACUUCGUCGAGUACGCUCAA-3′ |
| | (SEQ ID NO: 39) |

The coupling of a phosphoroimidazolide derived from a ribonucleoside 5′-monophosphate (e.g., guanosine 5′-monophosphate imidazolide, imGMP) to an oligonucleotide 5′-monophosphate (e.g., 5′-p-AUAGAACUUCGUCGAGUACGCUCAA-3′ (SEQ ID NO:24)) resulted in a nucleoside 5′-5′-diphosphate capped oligonucleotide (e.g., G(5′)-pp-(5′)AUAGAACUUCGUCGAGUACGCUCAA-3′ (SEQ ID NO:25)). The coupling of a phosphoroimidazolide derived from a ribonucleoside 5′-diphosphate (e.g., guanosine 5′-diphosphate imidazolide, imGDP) to an oligonucleotide 5′-monophosphate (e.g., 5′-p-AUAGAACUUCGUCGAGUACGCUCAA-3′ (SEQ ID NO:24)) resulted in a nucleoside 5′-5′-triphosphate capped oligonucleotide (e.g., G(5′)-ppp-(5′)AUAGAACUUCGUCGAGUACGCUCAA-3′ (SEQ ID NO:26)). The coupling of a phosphoroimidazolide derived from a ribonucleoside 5′-triphosphate (e.g., guanosine 5′-triphosphate imidazolide, imGTP) to an oligonucleotide 5′-monophosphate (e.g., 5′-p-AUAGAACUUCGUCGAGUACGCUCAA-3′ (SEQ ID NO:24)) resulted in a nucleoside 5′-5′-tetraphosphate capped oligonucleotide (e.g., G(5 ‘)-pppp-(5’)AUAGAACUUCGUCGAGUACGCUCAA-3′ (SEQ ID NO:27)). Table 4 and FIG. 2A-2F show some examples of nucleoside 5′-capped oligonucleotides obtained using this chemical approach (for simplicity, oligonucleotide IDs from Table 3 were used to represent individual oligonucleotide 5′-monophosphate sequences). In all cases, capped products comprise of a 5′-5′ polyphosphate linkage. Further examples of nucleoside 5′-phosphate activation chemistries for adding a desired nucleoside 5′ phosphate to the 5′ phosphate terminus of an RNA are shown in FIG. 1A-1B.

TABLE 4
Examples of nucleoside 5′-capped oligonucleotides obtained through chemical
capping (oligonucleotide sequence is represented by the first two nucleotides)

| | Oligonucleotide 5′-Monophosphate Sequence ID | | | |
|---|---|---|---|---|
| 5′-Phosphoroimidazolide | GU | AU | CU | UU |
| imGMP | GppGU | GppAU | GppCU | GppUU |
| imCMP | CppGU | CppAU | CppCU | n.a. |
| imAMP | AppGU | AppAU | AppCU | n.a. |
| imUMP | UppGU | UppAU | UppCU | UppUU |
| imGDP | GpppGU | GpppAU | GpppCU | GpppUU |
| imCDP | CpppGU | CpppAU | CpppCU | n.a. |
| imADP | ApppGU | ApppAU | ApppCU | n.a. |
| imUDP | UpppGG | UpppAU | UpppCU | n.a. |
| imIDP | IpppGG | IpppAU | IpppCU | IpppUU |
| imGTP | GppppGU | GppppAU | GppppCU | GppppUU |
| imCTP | CppppGU | CppppAU | n.a. | n.a. |
| imATP | AppppGU | AppppAU | n.a. | n.a. |
| imUTP | UppppGU | UppppAU | n.a. | n.a. |

n.a. indicates that the coupling reaction has not been attempted.

The chemical capping conditions were varied to in order to increase the reaction yields (FIG. 3B-3E). The coupling between a guanosine 5′-monophosphate imidazolide (imGMP) and an oligonucleotide 5′-monophosphate was evaluated in pHs ranging from 6 to 8 (FIG. 3B), in increasing molar excess of imGDP from 5× to 1000× (FIG. 3C), in increasing reaction times at 37° C. (FIG. 3D) and 50° C. (FIG. 3E).

The results shown in FIG. 3B-3C demonstrate that (i) pH 6 buffer instead of pH 7 buffer led to a yield increase from 31% to 63%; and (ii) the use of a higher molar excess of imidazolide reagent (im-GDP) from 10× to 1000× led to a yield increased from 12% to 73%. When the capping reaction was performed at 37° C. in pH 6 buffer using 1000×imGDP, about 85% yield was obtained in 4 hours and 91% yield in 6 hours (FIG. 3D). When the capping reaction was performed at 50° C. in pH 6 buffer using 1000×imGDP, about 62% yield was obtained in 1 hour or 68% yield in 2 hours (FIG. 3E).

The method as described in this example provided an 85% capping yield in 4 hours or 91% in 6 hours at 37° C.

### Example 4. Synthesis of Control Capped Polynucleotides by Enzymatic 7-Methylguanosine 5′-Capping of RNAs

Various 5′-capped oligonucleotide 25mer RNAs with m⁷G modifications were constructed as shown in Table 5.

Four preparations of 5′-triphosphate RNAs (5′-ppp-NUAGAACUUCGUCGAGUACGCUCAA-3′ (SEQ ID NO:40), wherein N was G, C, A, or U) were obtained according to Goldeck, et al. Angew. Chem. Int. Ed. 53, 4694-4698 (2014). GTP, dGTP, G_{2′F}TP, and AraGTP were purchased from TriLink Biotechnologies, San Diego, Calif. G_{3′Pr}TP and G_{3′DTB}TP were synthesized according to Ettwiller, et al. BMC Genomics, 17, 199 (2016).

Enzymatic capping of 5′-triphosphate RNAs (5 nmol) was performed at a 500 μL reaction using the Vaccinia Capping System (New England Biolabs, Ipswich, Mass.) supplemented with yeast inorganic pyrophosphate (New England Biolabs, Ipswich, Mass.) as follows: 10 μM 5′-triphosphate RNA, 1× Capping Buffer, 30 μM GTP or 500 μM GTP analog (dGTP, G_{2′F}TP, G_{3′Pr}TP, G_{3′DTB}TP, or AraGTP), 200 μM S-adenosylmethionine (SAM), 50 μL of pyrophosphate (0.1 unit/μL), and 50 μL of Vaccinia Capping Enzyme (1 unit/tit) were incubated at 37° C. overnight. For the synthesis of the 5′-capped oligonucleotides m⁷GpppNm, 2500 units of mRNA Cap 2′-O-Methyltransferase (New England Biolabs, Ipswich, Mass.) were included in the reaction. 7-Methylguanosine 5′-capped oligonucleotides were purified by phenol/chloroform extraction followed by polyacrylamide gel electrophoresis. The identity of the 5′-capped 25mer RNAs was confirmed by mass spectrometry (Oligo HTCS, Novatia, Newtown, Pa.).

TABLE 5
Examples of 7-methylguanosine 5′-capped oligonucleotides obtained through enzymatic
capping (oligonucleotide sequence is represented by the first nucleotide)

| | Cap 2′-O- | | | | |
|---|---|---|---|---|---|
| | Methyl | Oligonucleotide 5′-Triphosphate Sequence ID | | | |
| NTP | Transferase | G | A | C | U |
| GTP | − | m⁷GpppG | m⁷GpppA | m⁷GpppC | m⁷GpppU |
| GTP | + | m⁷GpppGm | m⁷GpppAm | m⁷GpppCm | m⁷GpppUm |
| dGTP | − | m⁷dGpppG | m⁷dGpppA | m⁷dGpppC | m⁷dGpppU |
| G_{2′F}TP | − | m⁷G_{2′F}pppG | m⁷G_{2′F}pppA | m⁷G_{2′F}pppC | m⁷G_{2′F}pppU |
| G_{3′Pr}TP | − | m⁷G_{3′Pr}pppG | m⁷G_{3′Pr}pppA | m⁷G_{3′Pr}pppC | m⁷G_{3′Pr}pppU |
| G_{3′DTB}TP* | − | G_{3′DTB}pppG | G_{3′DTB}pppA | G_{3′DTB}pppC | G_{3′DTB}pppU |
| AraGTP | − | m⁷AraGpppG | m⁷AraGpppA | m⁷AraGpppC | m⁷AraGpppU |

m⁷G represents 7-methylguanosine, N represents the first nucleotides of the oligonucleotide 25mer RNA sequence; Nm represents a nucleotide comprising an 2′-O-methylation; dG represent a 2′-deoxyriboguanosine; G_{2′F} represents a 2′-deoxy-2′-fluoroguanosine, G_{3′Pr} represents a 3′-O-propargylguanosine, G_{3′DTB} represents a 3′-O-desthiobiotinylatedguanosine, and AraG represents an araguanosine).

### Example 5. Evaluation of the Template Switching Ability of Viral Reverse Transcriptases

Four different Moloney Murine Leukemia Virus (MMLV)-type RTs were evaluated for their TS ability: SuperScript II, Maxima™ H Minus (Thermo Fisher Scientific, Waltham, Mass.), SMARTScribe, and Template Switching Reverse Transcriptase (here abbreviated as TS RT (New England Biolabs, Ipswich, Mass.).

The RNA templates in this example have a general sequence m⁷Gppp-NUAGAACUUCGUCGAGUACGCUCAA (SEQ ID NO:41), with N=G, C, A, or U.

Reverse transcription reactions were performed using a 5′-FAM DNA primer (see Table 1) which was complementary to the 3′ end of the RNA templates. The TSO was a hybrid oligonucleotide with 39 DNA nucleotides at the 5′-end and three RNA nucleotides at the 3′-end which were rGrGrG-3′ TSO (Table 1).

The TS reaction (10 μL total volume) was performed with 0.5 μM RNA template (2 μL, 100 nM final concentration), TS RT Buffer (2 μL), dNTP mix (1 μL, 1 mM final concentration of each dATP, dCTP, dGTP and dTTP (New England Biolabs, Ipswich, Mass.)), 1 μM primer (0.3 μL, 30 nM final concentration), 10 μM rGrGrG-3′ TSO (1 μL, 1 μM final concentration), water (3.2 μL), and a RT (0.5 μL). The reactions were performed at 42° C. for 90 minutes and were followed by a 10 minute heat-denaturation step at 72° C. The TS reactions were directly analyzed by capillary electrophoresis.

Capillary electrophoresis was performed as follows: 1 μL of a 1-20 nM sample was added to 10 μL of a mixture of HiDi™ Formamide (Thermo Fisher Scientific, Waltham, Mass.) and GeneScan™-120 LIZ™ Size Standard (1 μl of size standard to every 40 μl of formamide) (Thermo Fisher Scientific, Waltham, Mass.). The instrument used was either an Applied Biosystems 3130xl Genetic Analyzer (16 capillary array) or an Applied Biosystems 3730xl Genetic Analyzer (96 capillary array) with 36-cm long capillary coated with POP7 polymer. Data was collected via Applied Biosystems Data Collection software and analyzed with Applied Biosystems Peak Scanner software (Applied Biosystems, Foster City, Calif.).

As shown in FIG. 6, the TS efficiencies within a given RNA template were found to be similar across various MMLV-type RTs, except for Maxima H Minus RT that had an overall lower TS activity.

### Example 6. Evaluation of the Efficiency and Biases of Template Switching Reactions with Synthetic RNA Template Oligonucleotides

TS RT from New England Biolabs, Ipswich, Mass., was used throughout this study. The reverse transcription reactions were performed in the presence of a 5′-FAM DNA primer and a rGrGrG-3′ TSO (DNA primer and TSO sequences are shown in Table 1). RNA templates with 5′ termini that were 5′-hydroxyl (5′-HO—N), 5′-monophosphate (5′-p-N), 7-methylguanosine 5′-capped (5′-m⁷GpppN), and guanosine 5′-capped (5′-GpppN) oligonucleotide 25mers were compared to determine which 5′ terminus performed better in otherwise identical TS reactions. The RNA template general sequence was 5′-NUAGAACUUCGUCGAGUACGCUCAA-3′ (SEQ ID NO:42), where N was G, C, A, or U at position 1. The TS reaction (10 tit total volume in 1×TS RT buffer) was performed with 0.1 μM RNA template, dNTP mix (1 mM of each dATP, dCTP, dGTP and dTTP), 30 nM primer, 1 μM rGrGrG-3′ TSO, and TS RT (0.5 μL). The reactions were performed at 42° C. for 90 minutes and were followed by a 10 minute heat-denaturation step at 72° C. The TS reactions were directly analyzed by capillary electrophoresis as described in Example 5.

The results shown in FIG. 5A demonstrated that TS was more efficient for guanosine 5′-capped RNA templates (5′-Gppp; here also sometimes referred as unmethylated Gppp) than for 7-methylguanosine 5′-capped RNA templates (5′-m⁷Gppp). Uncapped RNAs (5′-HO—N and 5′-p-N) were 2- to 5-fold less efficient under these conditions compared to capped RNAs. Within a given RNA template sequence differing only at the nucleotide at position 1 (relative to the 5′ end; note that the cap nucleotide itself is not counted for determining positions 1, 2, 3, etc.), the order of efficiency in TS reactions is as follows G>A˜C>U (i.e., RNA templates starting with a guanosine at position 1 are the most efficient at TS, whereas RNA templates starting with a uridine are the least efficient). In fact, RNAs starting with a uridine are barely detected if the template is uncapped (FIG. 5B). Remarkably, the presence of a unmethylated guanosine cap (5′-Gppp) significantly reduces TS sequence biases, providing a more accurate representation of template abundance ratios. The chemically attached guanosine cap therefore provides not only a substantial gain in TS efficiency relative to the parent uncapped RNA template, but also unexpectedly reduces the sequence-dependence bias of the TS reaction relative to the naturally occurring 7-methylguanosine caps.

### Example 7. The Effect of Nonequimolar dNTP Mixes in the Efficiency of Template Switching Reactions

Concentrations of dNTPs were assayed in a TS reaction to determine their optimal relative concentrations.

The reverse transcription reactions were performed in the presence of a 5′-FAM DNA primer. A rGrGrG-3′ TSO or a ^{2′F}rG^{2′F}rG^{2′F}rG-3′ TSO were used in these experiments. DNA primer and TSO sequences are shown in Table 1. 7-Methylguanosine 5′-capped (m⁷GpppN) oligonucleotide 25mers were used as RNA templates (wherein N represents G, C, A, or U in the sequence 5′-NUAGAACUUCGUCGAGUACGCUCAA-3′ (SEQ ID NO:42)).

The TS reaction (10 tit total volume) was performed with 0.1 μM RNA template, TS RT Buffer (2 μL), dNTP mix (1 mM of each dATP, dCTP, dGTP and dTTP; or 1 mM of each dATP, dGTP, and dTTP, and 10 mM dCTP; or 1 mM of each dCTP, dGTP, and dTTP, and 0.1 mM dATP; or further combinations as described in Tables 6 and 7), 1 μM primer, 1 μM TSO, and TS RT (0.5 tit). The reactions were incubated at 42° C. for 90 minutes and were followed by a 10 minute heat-denaturation step at 72° C. The TS reactions were directly analyzed by capillary electrophoresis as described in Example 5. Results from these experiments are shown in FIG. 7 and Tables 6 and 7.

TABLE 6
Efficiencies of TS reactions using an equimolar dNTP mix, or
nonequimolar dNTP mixes comprising either an excess of dCTP
(10X dCTP), or an undersupply of dATP (0.1X dATP), or a combination
thereof. The RNA template sequence is represented by identity
of the nucleotide N at position 1. All reactions were carried
out in the presence of the rGrGrG-3′ TSO.

| | m⁷GpppA | m⁷GpppC | m⁷GpppG | m⁷GpppU |
|---|---|---|---|---|
| Equimolar dNTP mix | 37% | 27% | 64% | 9% |
| 10X dCTP | 69% | 62% | 92% | 46% |
| 0.1X dATP | 46% | 29% | 70% | 17% |
| 10X dCTP/0.5X dATP | 66% | 59% | 81% | 43% |

TABLE 7
Efficiencies of TS reactions using an equimolar dNTP mix, or
nonequimolar dNTP mixes comprising either an excess of dCTP
(10X dCTP) or an undersupply of dATP (0.1X dATP). The RNA
template sequence is represented by identity of the nucleotide
N at position 1. All reactions were carried out in the presence
of the ^{2′F}rG^{2′F}rG^{2′F}rG-3′ TSO.

| | m⁷GpppA | m⁷GpppC | m⁷GpppG | m⁷GpppU |
|---|---|---|---|---|
| Equimolar dNTP mix | 73% | 37% | 83% | 15% |
| 10X dCTP | 92% | 80% | 87% | 58% |
| 0.1X dATP | 80% | 40% | 90% | 29% |

The results in Table 6 show that when a 10-fold excess dCTP (10×dCTP) was utilized in the reverse transcription reaction, the TS efficiency increased by 1.5 to 5-fold for capped RNAs depending on the starting nucleotide (see also FIG. 7). 5′-m⁷GpppU RNA, which typically exhibits poor TS, showed the highest increase in efficiency, bringing it to a level comparable to the other RNA templates. These results support the idea that the addition of non-templated deoxycytidines to the 3′ end of the cDNA strand favor the formation of a stronger, more stable interaction between the cDNA and TSO. Accordingly, the efficiency of TS can be further increased by deploying chemical modifications that strengthen nucleic acid double-strand stability (for example, the 2′-fluoro modification in the ^{2′F}rG^{2′F}rG^{2′F}rG-3′ TSO) favoring the interaction of the cDNA with the TSO (Table 7).

The reverse transcription reaction carried out with a nonequimolar dNTP mixture comprising 1/10 of dATP (0.1×dATP) produces a smaller but distinguishable increase in the TS efficiency across all RNA templates. The use of different dNTP compositions and combinations thereof was also accessed (e.g., a 10×dCTP/0.5×dATP combination, FIG. 7).

### Example 8. Evaluation of Template Biases in a Sequencing Library Preparation from a Pool of Discrete and Equimolar Synthetic Oligonucleotides

Minimizing biases in next generation sequencing (NGS) polynucleotide library preparation is desirable for the generation of reliable sequencing data. An equimolar pool comprising the 16 synthetic 5′-monophosphate 25mer RNAs from Table 3 was prepared to permit collective evaluation of TS biases due to the first two nucleotides (positions 1 and 2) at the 5′ end of capped and uncapped RNA templates. The template pool was prepared by mixing equal amounts of these 16 discrete sequences and the sequences were confirmed by mass spectrometry.

The capping reaction of the RNA pool (20 μM final concentration) was carried out according to Example 3 at 37° C. for 4 hours in a 60 tit reaction. The reaction was diluted with water (180 μL) incubated with XRN-1 (5.5 tit) (New England Biolabs, Ipswich, Mass.) for 1 hour at 37° C. (the treatment with XRN-1 is optional; it can be used to degrade any remaining uncapped sequences in the template pool). The capping reaction products were purified with an Oligo Clean-up and Concentration Kit (Norgen Biotek, Ontario, Canada).

A TS RT reaction (16.5 μL total volume in 1×TS RT buffer) was performed as follows: A mixture of 0.1 μM RNA template, dNTP mix (1 mM of each dATP, dCTP, dGTP and dTTP), 30 nM i7 primer, 1 μM of i5-rGrGrG-3′ TSO was pre-incubated at 50° C. for 10 minutes, and then allowed to cool down slowly to room temperature. To this mixture was added BsrDI (1 μL) (New England Biolabs, Ipswich, Mass.), RNAse inhibitor, Murine (0.5 μL) (New England Biolabs, Ipswich, Mass.), and TS RT (2 tit) (New England Biolabs, Ipswich, Mass.). The reaction was performed at 42° C. for 90 minutes and was followed by a 10 minute heat-denaturation step at 72° C. Then 1 μL of the RT reaction was subjected to PCR amplification using Universal and Index primers using NEBNext® High-Fidelity 2×PCR Master Mix (New England Biolabs, Ipswich, Mass.). The libraries were cleaned up with NEBNext Sample Purification Beads (New England Biolabs, Ipswich, Mass.) and sequenced on an Illumina MiSeq or NextSeq. Libraries containing the 16 RNA pool were sequenced with the addition of 30% PhiX Control spike-in (Illumina, San Diego, Calif.). Adaptors were trimmed using Cutadapt and mapped to their respective sequences before being counted using BBMap.

Adding an unmethylated guanosine cap to the pool of template RNA to form guanosine 5′-capped RNAs (5′-GpppNN) was effective at significantly reducing biases of the TS reaction caused by variations of base composition at position 1 and 2 on the template RNA at the 5′ end. As shown in FIG. 8A, an even sequencing coverage (within 2-fold of the expected value) was obtained for the pool of 16 capped templates comprising all possible nucleotide variations at position 1 and 2. Additional experiments showed that nucleotides at positions 3 and 4 do not seem to affect bias significantly. Comparatively, the TS reaction with the uncapped RNA template pool comprising of 5′-monophosphate RNAs (5′-pNN) under the same conditions showed a strong bias against sequences starting with uridine or adenosine (FIG. 8B; sequences starting with AA, AC, UG and UU were particularly underrepresented in the sequencing reads).

Furthermore, the TS reverse transcription of guanosine 5′-capped RNA templates outperformed a library preparation protocol using conventional 3′ and 5′ enzymatic ligation steps (NEXTflex™ Small RNA-Seq Kit V3 (Bioo Scientific, Austin, Tex.); the sequencing library was prepared according to the manufacturer's instructions). Using the commercial kit (see FIG. 8C-8D), significantly more variation away from the expect sequencing read counts is observed in the 3′ and 5′ ligation-based library preparation approach (sequences starting with AG, GG, GU and UG were particularly underrepresented, while the sequences starting with GA was particularly overrepresented in the sequencing reads).

### Example 9. Sequence Analysis of RNA Libraries Having Alternative Cap Structures

Chemical capping was further utilized to generate RNA templates with the general structure 5′-XpₘpNN, wherein NN represents the first two variable nucleotides at the 5′ end of the 25mer sequence 5′-p-NNAGAACUUCGUCGAGUACGCUCAA-3′ (SEQ ID NO:20); X is nucleotide such as guanosine (G), inosine (I), adenosine (A), cytidine (C) or uridine (U); and m=1, 2, or 3 is the number of phosphates added through the chemical capping reaction. In all cases, capped products comprise of a 5′-5′ polyphosphate linkage.

The pool of 16 oligonucleotide 5′-monophosphate 25mer RNAs was first chemically capped with a given nucleoside 5′-phosphoroimidazolide of Table 2, followed by an optional step comprising treating the reaction mixture with XRN-1. Chemical capping of the RNA templates was performed as described in Example 8. The capping reaction was either at 50° C. for 5 hours (imNMPs), 37° C. for 4 hours (imNDPs), or room temperature for 4 hours (imNTPs). The chemically 5′-capped oligonucleotide pool was then reverse transcribed under the TS conditions of Example 8. PCR amplification and barcoding of the resulting cDNA library as well as library sequencing and analysis were performed according to Example 8.

Representative results are shown in FIG. 9. They confirm that the unmethylated guanosine cap forming a 5′-5′ triphosphate linkage with the RNA template (5′-GpppNN) provides the most uniform and accurate distribution of sequencing reads. An inosine cap forming a 5′-5′ triphosphate linkage with the RNA template (5′-IpppNN), and unmethylated guanosine caps forming a 5′-5′ diphosphate (5′-GppNN) or a 5′-5′ tetraphosphate (5′-GppppNN) linkage also produced accurate sequencing data.

### Example 10. Sequencing Library Preparation from an Equimolar Pool of 962 Unique miRNAs

A universal reference of 962 single-stranded synthetic 5′-monophosphate RNA oligonucleotides matching mature microRNAs (miRXplore, Miltenyi Biotec, Bergisch Gladbach, Germany) was utilized to demonstrate that a method comprising chemical capping and TS reverse transcription can be advantageously employed to generate cDNA libraries from a large collection of RNA templates.

Chemical capping of the miRXplore 962 unique miRNAs with guanosine 5′-diphosphate imidazolide (imGDP) was performed as described in Example 8. Purified guanosine 5′-capped (5′-Gppp) miRNAs were ligated either to a 3′-SR Adaptor (NEBNext Multiplex Small RNA Library Prep Set for Illumina, a 3′-Random Adaptor (Fuchs, et al. (2015) PLoS ONE 10(5):e0126049), or a 3′-Splint Adaptor (U.S. provisional patent application Ser. No. 62/839,191). The sequences of 3′-SR Adaptor, 3′-Random Adaptor, 3′-Splint Adaptor, and SR RT Primer are shown in Table 1. These three sets of 3′ adaptor ligated miRNAs were independently reverse transcribed under the TS conditions of Example 8 (1 μL SR RT Primer was used for reverse transcription of templates ligated to either 3′-SR Adaptor or 3′-Random Adaptor; USER Enzyme was used to generate the corresponding primer for reverse transcription of templates ligated to 3′-Splint Adaptor). PCR amplification and barcoding of the resulting cDNA libraries as well as library sequencing and analysis were performed according to Example 8. For comparison purposes, the miRXplore 962 unique miRNAs were subject to cDNA library preparation using a commercially available kit (TruSeq Small RNA Library Prep Kit) according to the manufacturer's instructions.

The results shown in FIG. 10A-10B demonstrate that miRNAs that have been capped with a guanosine (5′-Gppp) are substantially less prone to biases than those obtained from conventional small RNA library preparation. Guanosine 5′-capped miRNAs ligated to a 3′-SR Adaptor, a 3′-Random Adaptor, or a 3′-Splint Adaptor resulted in cDNA libraries where 45%-78% of the sequencing reads were within an interval of 2-fold from the expected value. On the other hand, cDNA libraries prepared with TruSeq Small RNA Library Prep Kit resulted in only 22% of the sequencing reads within an interval of 2-fold from the expected value. The latter resulted in a large number of underrepresented miRNA sequences, as evidenced in FIG. 10B. Furthermore, the relative variability in the datasets, calculated as the ratio of standard deviation to the mean (also known as the coefficient of variation, CV), was similar in the libraries obtained from guanosine 5′-capped miRNAs (CV in the range of 0.54 to 1.0), indicating a comparable performance. In contrast, the TruSeq libraries showed a significantly higher variance (CV approximately 2.6).

### Example 11. Sequencing Library Preparation from a Non-Equimolar Pool of 12 Unique Synthetic miRNAs in a Total RNA Background

A pool of 12 discrete synthetic miRNAs (here named as Mix4v7), where the relative input of individual sequences ranged from 1- to 250-fold, was combined with a sample of total RNA extracted from human adult normal brain tissue (Biochain, Newark, Calif.), and cDNA libraries were generated from the mixture by means of chemical capping and TS reverse transcription. The sequences and relative amounts of individual miRNAs are shown in Table 8.

TABLE 8
Mix4v7 Synthetic miRNAs.

| | SEQ | | Relative |
|---|---|---|---|
| ID | ID NO | Sequence | Amount |
| 26e spike | 43 | CAAGUUUUAAUACUUAUUAGUAU | 2 |
| 32c spike | 44 | UUUCAUAGUACUUUAGUAACAUG | 16 |
| 32g spike | 45 | GCAUGCAUAGAUUCUUAAUAUUU | 31.25 |
| 38z spike | 46 | UUAUGAAGGACGCAUCAUUUUAC | 4 |
| 38c spike | 47 | UAUCUUUAGCGCCUAGUUAAAAG | 125 |
| 44d spike | 48 | AUCCGAUUACGACGUGUACGUUA | 4 |
| 50g spike | 49 | UAUUUGGACUUAACCAGCCUGUG | 2 |
| 56z spike | 50 | GUAGGCUAACCUGACGGCUACUU | 1 |
| 62b spike | 51 | AUCCGUGGCGCGGCAUCAUCUAG | 8 |
| 68b spike | 52 | CUAGCCGCCUGGCUAUAGGGUUC | 250 |
| 74a spike | 53 | AGCGUGCGUCCGUGGCCAUCUCG | 62.5 |
| 74c spike | 54 | UCGCGCGCGGCUCGUAACAGCGU | 1 |

The 5′-monophosphorylated Mix4v7 miRNA spike-in human brain RNA was subjected to chemical capping with guanosine 5′-diphosphate imidazolide (imGDP) as described in Example 8. Purified 5′-Capped (5′-Gppp) RNAs were ligated either to a 3′-SR Adaptor, a 3′-Random Adaptor, or a 3′-Splint Adaptor and independently reverse transcribed according to protocols described in Example 10. PCR amplification and barcoding of the resulting cDNA libraries as well as library sequencing and analysis were performed according to Example 8.

The results shown in FIG. 11 demonstrate that the normalized sequencing reads derived from guanosine 5′-capped (5′-Gppp) miRNAs are largely independent of the relative frequency of individual miRNAs. Furthermore, no apparent correlation was observed regarding read rates and amounts of miRNA sequences in the sample. Thus, unbiased sequencing of a population of chemically capped (unmethylated guanosine) RNAs, can be achieved where lack of bias is measured by capture of all sequences within a 2-fold band around the theoretical value.

### Example 12. Sequencing Library Preparation from Samples of Total RNA from Human Brain, Heart and Liver Tissues

The method comprising chemical capping and TS reverse transcription was extended to the preparation of cDNA libraries from various total RNA samples, including total RNAs extracted from human adult normal brain tissue, from human adult normal brain heart tissue, and from human adult normal liver formalin-fixed paraffin-embedded (FFPE) tissue. Prior to chemical capping, total RNA samples were pre-treated with T4 Polynucleotide Kinase (T4 PNK) to phosphorylate RNA 5′ ends and to remove phosphoryl groups (including terminal 2′,3′-cyclic phosphates) from RNA 3′ ends.

Phosphorylation of human brain total RNA (1 μg) (Biochain, Newark, Calif.) or human heart total RNA (1 μg) (Biochain, Newark, Calif.) or human liver FFPE total RNA (0.2 μg) (Biochain, Newark, Calif.) was performed by combining total RNA sample with water (up to final volume of 50 tit), 10×T4 PNK Reaction Buffer (New England Biolabs, Ipswich, Mass.), ATP (final concentration 1 mM) (New England Biolabs, Ipswich, Mass.), and T4 PNK (1 μL). The reaction was incubated for 30 minutes at 37° C. after which time the material was subjected to purification using the Monarch® RNA Cleanup Kit (New England Biolabs, Ipswich, Mass.) following the protocol for isolation of small RNAs from large RNAs.

The purified 5′-monophosphorylated human brain, heart or liver RNA was subjected to chemical capping with guanosine 5′-diphosphate imidazolide (imGDP) as described in Example 8. Purified guanosine 5′-capped (5′-Gppp) RNAs were ligated either to a 3′-Random Adaptor or a 3′-Splint Adaptor and independently reverse transcribed according to protocols described in Example 10. PCR amplification and barcoding of the resulting cDNA libraries as well as library sequencing and analysis were performed according to Example 8.

The results in FIG. 12A-12C show the most abundant miRNAs identified in the human brain, heat and liver total RNA samples, demonstrating that the method comprising chemical capping and TS reverse transcription can be used with different types of biological samples (total RNA is isolated from tissues by modified guanidine thiocyanate techniques and stored in RNA storage; FFPE Total RNA is isolated from formalin-fixed paraffin-embedded materials). Of importance, the method is suitable to FFPE samples (FIG. 12C). The combination of chemical capping and TS reverse transcription can be advantageously employed for making cDNA libraries from FFPE samples, as this enables the detection of any nucleic acid fragments (including fragmented mRNAs) provided the 5′ and 3′ ends have been repaired with an enzyme such as T4 PNK. PNK phosphorylates DNA or RNA 5′ ends and removes phosphoryl groups from DNA or RNA 3′ ends.

### Example 13. cDNA Sequencing Libraries Prepared by Template Switching from Single-Stranded DNA Templates

Chemical capping and TS reverse transcription was used to generate cDNA libraries from DNA templates. A non-equimolar pool of 16 discrete ssDNA templates was obtained where the relative input of individual sequences ranged from 1- to 250-fold. The sequences and relative amounts of individual ssDNA are shown in Table 9.

TABLE 9
Synthetic ssDNAs.

| | SEQ ID | | Relative |
|---|---|---|---|
| ID | NO | Sequence | Amount |
| 26e | 55 | d(CAAGTTTTAATACTTATTAGTAT) | 2 |
| 32c | 56 | d(TTTCATAGTACTTTAGTAACATG) | 16 |
| 32g | 57 | d(GCATGCATAGATTCTTAATATTT) | 31.3 |
| 38z | 58 | d(TTATGAAGGACGCATCATTTTAC) | 4 |
| 38c | 59 | d(TATCTTTAGCGCCTAGTTAAAAG) | 125 |
| 44d | 60 | d(ATCCGATTACGACGTGTACGTTA) | 4 |
| 50g | 61 | d(TATTTGGACTTAACCAGCCTGTG) | 2 |
| 56z | 62 | d(GTAGGCTAACCTGACGGCTACTT) | 1 |
| 62b | 63 | d(ATCCGTGGCGCGGCATCATCTAG) | 8 |
| 68b | 64 | d(CTAGCCGCCTGGCTATAGGGTTC) | 250 |
| 74a | 65 | d(AGCGTGCGTCCGTGGCCATCTCG) | 62.5 |
| 74c | 66 | d(TCGCGCGCGGCTCGTAACAGCGT) | 1 |
| 50e | 67 | d(GTAGGTTCATATTCTCTAGGCAC) | 62.5 |

| | |
|---|---|
| | 3′-Splint Adaptor |
| | (for DNA templates) |
| 18 | 5′-p-d(AGATCGGAAGAGCACACGTCT)- |
| | ddC-3′ |
| 19 | 3′-NH2-d(NNNNNNUCTAGCCTTCTCGTG |
| | TGCAGA)-5′ |

The capping reaction was performed as described in Example 8. A pool of sixteen 5′-monophosphate ssDNAs (20 μM final concentration) to final volume of 60 μL in a chemical capping buffer pH 6.0 containing an organic cosolvent was combined with 30 mM guanosine 5′-diphosphate imidazolide (imGDP), and the capping reaction incubated at 37° C. for 4 hours. The capping reaction products were purified with an Oligo Clean-up and Concentration Kit.

3′ Adaptors were added to guanosine 5′-capped (5′-Gppp) or uncapped (5′-p) ssDNA templates by splint ligation as described by U.S. Provisional Application No. 62/839,191.

For capillary electrophoresis analysis, 5′-capped (5′-Gppp) or uncapped (5′-p) ssDNA templates were reverse transcribed in the presence of 10 μM 5′-FAM SR RT primer (1.25 μL; primer sequence 5′-FAM-AGACGTGTGCTCTTCCGATCT-3′ (SEQ ID NO: 68)) as described in Example 5. For deep sequencing analysis, the 5′-FAM SR RT primer was omitted from the TS reverse transcription reaction. PCR amplification and barcoding of the resulting cDNA libraries as well as library sequencing and analysis were performed according to Example 8.

The results in FIG. 13A-13C are consistent with those for RNA templates. The results demonstrate that the method comprising chemical capping and TS reverse transcription can indeed be used for the generation of cDNA libraries from DNA templates. FIG. 13A shows that TS is more efficient with guanosine 5′-capped (5′-Gppp) ssDNA templates than with uncapped (5′-p) ssDNA templates under the same conditions. Furthermore, as shown in FIG. 13B, a less biased sequencing coverage is obtained for guanosine 5′-capped (5′-Gppp) ssDNA templates than is for uncapped (5′-p) ssDNA templates. The analysis of ssDNA by TS finds use in preparing sequence libraries of degraded and/or fragmented DNA such as ancient DNA, environmental DNA, forensic DNA, circulating DNA (e.g., exosomes), denatured DNA and viral DNA. Several of these DNAs may be used as biomarkers and in medical diagnosis applications.

1. A method for chemically capping a population of polynucleotides having a 5′ monophosphate, comprising:
(a) combining an activated nucleoside 5′ mono-phosphate or an activated nucleoside 5′ poly-phosphate with the population of polynucleotides, to produce a reaction mix; and
(b) incubating the reaction mix to produce reaction products that each comprise a polynucleotide linked to a 5′ nucleoside cap by a 5′ to 5′ polyphosphate linkage.

2. The method according to claim 1, wherein the population of polynucleotides are RNAs.

3. The method of claim 2, wherein the RNA population comprises one or more of RNA species selected from the group consisting of small RNAs, microRNAs, tRNAs, long noncoding RNAs, and fragmented mRNAs.

4. The method according to claim 1, wherein the polynucleotides in the population are single-stranded or partially single-stranded DNAs.

5. The method of claim 1, wherein the population of polynucleotides is a population of polynucleotides of a cell, a population of polynucleotides of a virus, a population of polynucleotides of a liquid biopsy or a population of polynucleotides of a formalin-fixed, paraffin-embedded tissue.

6. The method according to claim 1, wherein the polynucleotides having a 5′ monophosphate sis produced by enzymatically adding a single phosphate to the 5′ end of polynucleotides that have no terminal phosphate.

7. The method according to claim 6, wherein the single phosphate is added using a polynucleotide kinase.

8. The method according to claim 1, further comprising decapping 5′ capped RNAs to produce the polynucleotides having a 5′ monophosphate.

9. The method according to claim 8, wherein the decapping further comprises contacting the 5′ capped RNAs with an enzyme selected from a deadenylase, an apyrase, a 5′RNA polyphosphatase (RppH), an Nudix phosphohydrolase, a tobacco acid polyphosphatase, a member of the histidine triad (HIT) superfamily of pyrophosphatases, a DcpS, a Dcp1-Dcp2 complex, a NudC, or an aprataxin (APTX).

10. The method according to claim 1, wherein the activated nucleoside 5′ mono-phosphate or the activated nucleoside 5′ poly-phosphate comprises an imidazole and the incubating the reaction mix further comprises incubating the reaction mix to permit a nucleophilic substitution reaction that displaces the imidazole.

11. The method according to claim 10, wherein the reaction mix has a pH of in the range of pH 5-pH 6.5.

12. The method according to claim 11, wherein the incubating the reaction mix further comprises incubating the reaction mix for less than 10 hours at a temperature of less than 60° C.

13. The method according to claim 1,
(i) wherein the activated nucleoside 5′ mono- or poly-phosphate of (a) is an imidazolide nucleoside 5′-monophosphate, 5′-diphosphate or 5′ triphosphate and the incubating the reaction mix further comprises incubating the reaction mix either at 50° C. for 5 hours, 37° C. for 4 hours, or room temperature for 4 hours, and
(ii) further comprising (c) displacing the imidazole to form the 5′ capped polynucleotides.

14. The method according to claim 3, wherein the 5′ nucleoside cap is of formula (I): 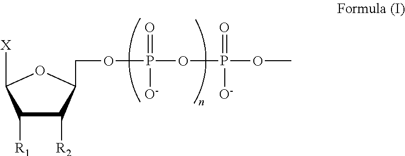
wherein, X is a nitrogenous base; R1 and/or R2═O-alkyl, halogen, a linker, hydrogen or a hydroxyl; n is any integer from 1-9; and the polynucleotide cap is a single stereoisomer or plurality of stereoisomers of one or more of the compounds described by Formula (I) or a salt or salts thereof.

15. The method according to claim 14, wherein the nitrogenous base of the 5′ nucleoside cap is selected from the group consisting guanine, adenine, cytosine, uracil and hypoxanthine and analogs of guanine, adenine, cytosine, uracil and hypoxanthine.

16. The method of claim 14, wherein the nitrogenous base of the 5′ nucleoside cap comprises a modified base selected from N6-methyladenine, N1-methyladenine, N6-2′-O-dimethyladenosine, pseudouridine, N1-methylpseudouridine, 5-iodouridine, 4-thiouridine, 2-thiouridine, 5-methyluridine, pseudoisocytosine, 5-methoxycytosine, 2-thiocytosine, 5-hydroxycytosine, N4-methylcytosine, 5-hydroxymethylcytosine, hypoxanthine, N1-methylguanine, O6-methylguanine, 1-methyl-guanosine, N2-methyl-guanosine, N7-methyl-guanosine, N2,N2-dimethyl-guanosine, 2-methyl-2′-O-methyl-guanosine, N2,N2-dimethyl-2′-O-methyl-guanosine, 1-methyl-2′-O-methyl-guanosine, N2,N7-dimethyl-2′-O-methyl-guanosine, or isoguanine.

17. The method of claim 14, wherein the nitrogenous base of the 5′ nucleoside cap is attached to a sugar consisting of a ribose or a modified ribose selected from 2′- or 3′-O-alkylribose, alkoxyribose, O-alkoxyalkylribose, fluororibose, azidoribose, allylribose, deoxyribose; an arabinose or a modified arabinose; a thioribose; an 1,5 anhydrohexitol; or a threofuranose.

18. The method of claim 14, wherein the one or more phosphates of the 5′ nucleoside cap consists of a phosphorothioate; a phosphorodithioate; an alkyphosphonate; an arylphosphonate; a N-phosphoramidate; a boranophosphate; or a phosphonoacetate.

19. The method according to claim 14, wherein the 5′ nucleoside cap comprises guanosine.

20. The method according to claim 14, wherein the polynucleotide further comprises a 3′ poly A tail or a 3′ ligated adapter on the 5′ capped polynucleotides for priming a reverse transcriptase; and (e) forming a cDNA.

21. The method according to claim 20, wherein the cDNA further comprises a sequence at the 3′ end that is complementary to a template switching oligonucleotide (TSO).

22. The method according to claim 14, further comprising amplifying the cDNA to produce an amplification product.

23. The method according to claim 21, further comprising sequencing the cDNA or amplification product thereof.

24. The method according to claim 19, wherein the cDNA corresponding to the population of polynucleotides is not substantially biased in favor of one or more of the following: (i) polynucleotides that have a specific nucleotide of the four nucleotides selected from the group consisting of A, G, C, and U or T in the first or second position at the 5′ end over any other of the four nucleotides or (ii) a cap comprising guanosine and one, two, three or four phosphates between the guanosine cap and the first nucleotide at the 5′ end.

25. The method according to claim 19, wherein the efficiency of template switching is enhanced by at least 2-fold compared with 5′ capped polynucleotides that do not comprise an unmethylated guanosine.

26. A method for synthesizing a cDNA from a single strand polynucleotide, comprising the steps of:
(a) combining an activated nucleoside 5′ mono- or poly-phosphate with a population of polynucleotides having a 5′ monophosphate, to produce a reaction mix;
(b) incubating the reaction mix to produce reaction products that each comprise a polynucleotide and a 5′ nucleoside cap, linked by a 5′ to 5′ polyphosphate linkage; and
(c) reverse transcribing the products of step (c) in the presence of template switching oligonucleotide (TSO) to produce cDNA that comprises the complement of the TSO at the cDNA 3′ end.

27. The method according to claim 26, wherein the method further comprises: (i) ligating a 3′ adaptor to the polynucleotides prior to step (a) or (ii) ligating a 3′ adaptor to the reaction products of (b), the adapter optionally contains a cDNA priming site, and wherein the reverse transcribing is done using a cDNA synthesis primer that hybridizes to the adapter.

28. The method according to claim 26; wherein the method comprises any of the methods of claims 1-26.

29. The method of claim 26, wherein the 5′ nucleoside cap is a guanosine triphosphate.

30. The method of claim 26, further comprising sequencing the cDNA.

31. The method according to claim 29, wherein the yield of cDNA having 5′ and 3′ adapter sequences that is the product of reverse transcription of the population of DNA having a 5′ cap guanosine triphosphate is increased at least 2-fold compared with the yield of cDNA product from a population of DNA that are not capped with a 5′ cap guanosine triphosphate.

32. (canceled)

33. (canceled)

34. (canceled)


SEQUENCE LISTING (68 sequences)

SEQ ID NO: 1 - DNA, 11 bp, Artificial Sequence
  Synthetic construct
aaaaaaaaaaa

SEQ ID NO: 2 - DNA, 20 bp, Artificial Sequence
  Synthetic construct
  misc_feature: 5′FAM DNA primer
  modified_base (1)..(1): 5′ carboxyfluorescein (FAM)
ttgagcgtactcgacgaagt

SEQ ID NO: 3 - DNA, 57 bp, Artificial Sequence
  Synthetic construct
  misc_feature: i7 Primer
  misc_feature (33)..(38): n is a, c, g, or t
gactggagttcagacgtgtgctcttccgatctnnnnnnttgagcgtactcgacgaag

SEQ ID NO: 4 - DNA, 33 bp, Artificial Sequence
  Synthetic construct
  misc_feature: Poly(dT) primer
  misc_feature (33)..(33): n is a, c, g, or t
agacgtgtgctcttccgatctttttttttttvn

SEQ ID NO: 5 - DNA, 21 bp, Artificial Sequence
  Synthetic construct
  misc_feature: SR RT primer
agacgtgtgctcttccgatct

SEQ ID NO: 6 - DNA, 42 bp, Artificial Sequence
  Synthetic construct
  modified_base (40)..(40): guanosine (ribonucleoside)
  modified_base (41)..(41): guanosine (ribonucleoside)
  modified_base (42)..(42): guanosine (ribonucleoside)
gctaatcattgcaagcagtggtatcaacgcagagtacatggg

SEQ ID NO: 7 - DNA, 42 bp, Artificial Sequence
  Synthetic construct
  modified_base (40)..(40): uridine (ribonucleoside)
  modified_base (41)..(41): uridine (ribonucleoside)
  modified_base (42)..(42): uridine (ribonucleoside)
gctaatcattgcaagcagtggtatcaacgcagagtacatuug

SEQ ID NO: 8 - DNA, 42 bp, Artificial Sequence
  Synthetic construct
  modified_base (40)..(40): guanosine (ribonucleoside)
  modified_base (41)..(41): guanosine (ribonucleoside)
  modified_base (42)..(42): guanosine (ribonucleoside)
gctaatcattgcaagcagtggtatcaacgcagagtacatgug

SEQ ID NO: 9 - DNA, 42 bp, Artificial Sequence; identical to SEQ ID NO: 6
  Synthetic construct
  modified_base (37)..(37): 2′-fluoro-guanosine (ribonucleoside)
  modified_base (38)..(38): 2′-fluoro-guanosine (ribonucleoside)
  modified_base (39)..(39): 2′-fluoro-guanosine (ribonucleoside)

SEQ ID NO: 10 - DNA, 49 bp, Artificial Sequence
  Synthetic construct
  modified_base (47)..(47): guanosine (ribonucleoside)
  modified_base (48)..(48): guanosine (ribonucleoside)
  modified_base (49)..(49): guanosine (ribonucleoside)
actaatcattgctacactctttccctacacgacgctcttccgatctggg

SEQ ID NO: 11 - DNA, 22 bp, Artificial Sequence
  Synthetic construct
  modified_base (1)..(1): 5′-pre-adenylated end (5′-App-)
  modified_base (22)..(22): 3′-NH2
aagatcggaagagcacacgtct

SEQ ID NO: 12 - DNA, 35 bp, Artificial Sequence
  Synthetic construct
  modified_base (1)..(1): 5′-pre-adenylated end (5′-App-)
  misc_feature (9)..(14): n is a, c, g, t or u
  modified_base (35)..(35): 3′-NH2
atcgtatgnnnnnnagatcggaagagcacacgtct

SEQ ID NO: 13 - DNA, 35 bp, Artificial Sequence
  Synthetic construct
  modified_base (1)..(1): 5′-pre-adenylated end (5′-App-)
  misc_feature (9)..(14): n is a, c, g, t or u
  modified_base (35)..(35): 3′-NH2
aagcatacnnnnnnagatcggaagagcacacgtct

SEQ ID NO: 14 - DNA, 35 bp, Artificial Sequence
  Synthetic construct
  modified_base (1)..(1): 5′-pre-adenylated end (5′-App-)
  misc_feature (9)..(14): n is a, c, g, t or u
  modified_base (35)..(35): 3′-NH2
actacgcannnnnnagatcggaagagcacacgtct

SEQ ID NO: 15 - DNA, 35 bp, Artificial Sequence
  Synthetic construct
  modified_base (1)..(1): 5′-pre-adenylated end (5′-App-)
  misc_feature (9)..(14): n is a, c, g, t or u
  modified_base (35)..(35): 3′-NH2
agatgcgtnnnnnnagatcggaagagcacacgtct

SEQ ID NO: 16 - DNA, 23 bp, Artificial Sequence
  Synthetic construct
  modified_base (1)..(1): 5′-pre-adenylated end (5′-App-)
  modified_base (23)..(23): dideoxynucleotide
aagatcggaagagcacacgtctc

SEQ ID NO: 17 - DNA, 27 bp, Artificial Sequence
  Synthetic construct
  modified_base (21)..(21): uridine (ribonucleoside)
  misc_feature (22)..(27): n is a, c, g, t or u
  modified_base (27)..(27): 3′-NH2
agacgtgtgctcttccgatcunnnnnn

SEQ ID NO: 18 - DNA, 22 bp, Artificial Sequence
  Synthetic construct
  misc_feature: 3′-Splint Adaptor
  modified_base (22)..(22): C-dideoxynucleotide
agatcggaagagcacacgtctc

SEQ ID NO: 19 - DNA, 27 bp, Artificial Sequence
  Synthetic construct
  modified_base (21)..(21): uridine (ribonucleoside)
  misc_feature (22)..(27): n is a, c, g, t or u
  modified_base (27)..(27): 3′-NH2
agacgtgtgctcttccgatcunnnnnn

SEQ ID NO: 20 - RNA, 25 nt, Artificial Sequence
  Synthetic construct
  misc_feature (1)..(2): n is a, c, g, or u
nnagaacuucgucgaguacgcucaa

SEQ ID NO: 21 - RNA, 25 nt, Artificial Sequence
  Synthetic construct
aaagaacuucgucgaguacgcucaa

SEQ ID NO: 22 - RNA, 25 nt, Artificial Sequence
  Synthetic construct
acagaacuucgucgaguacgcucaa

SEQ ID NO: 23 - RNA, 25 nt, Artificial Sequence
  Synthetic construct
agagaacuucgucgaguacgcucaa

SEQ ID NO: 24 - RNA, 25 nt, Artificial Sequence
  Synthetic construct
auagaacuucgucgaguacgcucaa

SEQ ID NO: 25 - RNA, 26 nt, Artificial Sequence
  Synthetic construct
  modified_base (1)..(1): Guanosine 5′-5′ diphosphate cap
gauagaacuucgucgaguacgcucaa

SEQ ID NO: 26 - RNA, 26 nt, Artificial Sequence
  Synthetic construct
  modified_base (1)..(1): Guanosine 5′-5′ triphosphate cap
gauagaacuucgucgaguacgcucaa

SEQ ID NO: 27 - RNA, 26 nt, Artificial Sequence
  Synthetic construct
  modified_base (1)..(1): Guanosine 5′-5′ tetraphosphate cap
gauagaacuucgucgaguacgcucaa

SEQ ID NO: 28 - RNA, 25 nt, Artificial Sequence
  Synthetic construct
caagaacuucgucgaguacgcucaa

SEQ ID NO: 29 - RNA, 25 nt, Artificial Sequence
  Synthetic construct
ccagaacuucgucgaguacgcucaa

SEQ ID NO: 30 - RNA, 25 nt, Artificial Sequence
  Synthetic construct
cgagaacuucgucgaguacgcucaa

SEQ ID NO: 31 - RNA, 25 nt, Artificial Sequence
  Synthetic construct
cuagaacuucgucgaguacgcucaa

SEQ ID NO: 32 - RNA, 25 nt, Artificial Sequence
  Synthetic construct
gaagaacuucgucgaguacgcucaa

SEQ ID NO: 33 - RNA, 25 nt, Artificial Sequence
  Synthetic construct
gcagaacuucgucgaguacgcucaa

SEQ ID NO: 34 - RNA, 25 nt, Artificial Sequence
  Synthetic construct
ggagaacuucgucgaguacgcucaa

SEQ ID NO: 35 - RNA, 25 nt, Artificial Sequence
  Synthetic construct
guagaacuucgucgaguacgcucaa

SEQ ID NO: 36 - RNA, 25 nt, Artificial Sequence
  Synthetic construct
uaagaacuucgucgaguacgcucaa

SEQ ID NO: 37 - RNA, 25 nt, Artificial Sequence
  Synthetic construct
ucagaacuucgucgaguacgcucaa

SEQ ID NO: 38 - RNA, 25 nt, Artificial Sequence
  Synthetic construct
ugagaacuucgucgaguacgcucaa

SEQ ID NO: 39 - RNA, 25 nt, Artificial Sequence
  Synthetic construct
uuagaacuucgucgaguacgcucaa

SEQ ID NO: 40 - RNA, 25 nt, Artificial Sequence
  Synthetic construct
  modified_base (1)..(1): 5′ triphosphate
  misc_feature (1)..(1): n is a, c, g, or u
nuagaacuucgucgaguacgcucaa

SEQ ID NO: 41 - RNA, 26 nt, Artificial Sequence
  Synthetic construct
  modified_base (1)..(1): 7-methylguanosine 5′-triphosphate cap
  misc_feature (2)..(2): n is a, c, g, or u
gnuagaacuucgucgaguacgcucaa

SEQ ID NO: 42 - RNA, 25 nt, Artificial Sequence
  Synthetic construct
  misc_feature (1)..(1): n is a, c, g, or u
nuagaacuucgucgaguacgcucaa

SEQ ID NO: 43 - RNA, 23 nt, Artificial Sequence
  Synthetic construct
caaguuuuaauacuuauuaguau

SEQ ID NO: 44 - RNA, 23 nt, Artificial Sequence
  Synthetic construct
uuucauaguacuuuaguaacaug

SEQ ID NO: 45 - RNA, 23 nt, Artificial Sequence
  Synthetic construct
gcaugcauagauucuuaauauuu

SEQ ID NO: 46 - RNA, 23 nt, Artificial Sequence
  Synthetic construct
uuaugaaggacgcaucauuuuac

SEQ ID NO: 47 - RNA, 23 nt, Artificial Sequence
  Synthetic construct
uaucuuuagcgccuaguuaaaag

SEQ ID NO: 48 - RNA, 23 nt, Artificial Sequence
  Synthetic construct
auccgauuacgacguguacguua

SEQ ID NO: 49 - RNA, 23 nt, Artificial Sequence
  Synthetic construct
uauuuggacuuaaccagccugug

SEQ ID NO: 50 - RNA, 23 nt, Artificial Sequence
  Synthetic construct
guaggcuaaccugacggcuacuu

SEQ ID NO: 51 - RNA, 23 nt, Artificial Sequence
  Synthetic construct
auccguggcgcggcaucaucuag

SEQ ID NO: 52 - RNA, 23 nt, Artificial Sequence
  Synthetic construct
cuagccgccuggcuauaggguuc

SEQ ID NO: 53 - RNA, 23 nt, Artificial Sequence
  Synthetic construct
agcgugcguccguggccaucucg

SEQ ID NO: 54 - RNA, 23 nt, Artificial Sequence
  Synthetic construct
ucgcgcgcggcucguaacagcgu

SEQ ID NO: 55 - DNA, 23 bp, Artificial Sequence
  Synthetic construct
caagttttaatacttattagtat

SEQ ID NO: 56 - DNA, 23 bp, Artificial Sequence
  Synthetic construct
tttcatagtactttagtaacatg

SEQ ID NO: 57 - DNA, 23 bp, Artificial Sequence
  Synthetic construct
gcatgcatagattcttaatattt

SEQ ID NO: 58 - DNA, 23 bp, Artificial Sequence
  Synthetic construct
ttatgaaggacgcatcattttac

SEQ ID NO: 59 - DNA, 23 bp, Artificial Sequence
  Synthetic construct
tatctttagcgcctagttaaaag

SEQ ID NO: 60 - DNA, 23 bp, Artificial Sequence
  Synthetic construct
atccgattacgacgtgtacgtta

SEQ ID NO: 61 - DNA, 23 bp, Artificial Sequence
  Synthetic construct
tatttggacttaaccagcctgtg

SEQ ID NO: 62 - DNA, 23 bp, Artificial Sequence
  Synthetic construct
gtaggctaacctgacggctactt

SEQ ID NO: 63 - DNA, 23 bp, Artificial Sequence
  Synthetic construct
atccgtggcgcggcatcatctag

SEQ ID NO: 64 - DNA, 23 bp, Artificial Sequence
  Synthetic construct
ctagccgcctggctatagggtcc

SEQ ID NO: 65 - DNA, 23 bp, Artificial Sequence
  Synthetic construct
agcgtgcgtccgtggccatctcg

SEQ ID NO: 66 - DNA, 23 bp, Artificial Sequence
  Synthetic construct
tcgcgcgcggctcgtaacagcgt

SEQ ID NO: 67 - DNA, 23 bp, Artificial Sequence
  Synthetic construct
gtaggttcatattctctaggcac

SEQ ID NO: 68 - DNA, 21 bp, Artificial Sequence
  Synthetic construct
  modified_base (1)..(1): 5′ carboxyfluorescein (FAM)
agacgtgtgctcttccgatct